(19) United States
(12) Patent Application Publication
(10) Pub. No.: US 2023/0106353 A1
(43) Pub. Date: Apr. 6, 2023

(54) SYSTEM FOR COVALENTLY LINKING PROTEINS

(71) Applicant: Oxford University Innovation Limited, Oxford (Oxfordshire) (GB)

(72) Inventors: Mark HOWARTH, Oxford (Oxfordshire) (GB); Arne Hagen August SCHEU, Oxford (Oxfordshire) (GB); Ying Ting Sheryl LIM, Oxford (Oxfordshire) (GB)

(21) Appl. No.: 17/910,847

(22) PCT Filed: Mar. 12, 2021
(86) PCT No.: PCT/GB2021/050625

§ 371 Date: Sep. 12, 2022

(30) Foreign Application Priority Data
Mar. 13, 2020 (GB) 2003683.6

(51) Int. Cl. C07K 14/22; A61K 47/62; C07K 1/14; C12N 9/88; C07K 14/495; C07K 14/71

(52) U.S. Cl. CPC C07K 14/22; A61K 47/62; C07K 1/145; C12N 9/88; C12Y 401/01017; C07K 14/495; C07K 14/71; A61K 38/00

(57) ABSTRACT

The present invention relates to a system for generating intermolecular covalent bonds (e.g. amide, e.g. isopeptide bonds) between polypeptides. In particular, it provides the use of a chimeric protein to generate an anhydride group on a polypeptide for the formation of a covalent bond, wherein the chimeric protein comprises (i) a domain comprising the polypeptide and (ii) a domain comprising a self-processing module that contains an N-terminal dipeptide of aspartate or glutamate and proline (D/E-P), wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue in the self-processing module to release the polypeptide and generate the anhydride group on the aspartate or glutamate residue.

The present invention relates to a system for generating intermolecular covalent bonds (e.g. amide, e.g. isopeptide bonds) between polypeptides, e.g. covalently linking polypeptides via an isopeptide bond, or intramolecular covalent bonds (e.g. amide, e.g. isopeptide bonds) within a polypeptide. In particular, the system utilises a chimeric polypeptide comprising a self-processing module that undergoes autoproteolysis to generate a first polypeptide (e.g. binding polypeptide) comprising an electrophile (e.g. an anhydride group) that can react specifically with a nucleophile (e.g. an amine group) within the first polypeptide or on a second polypeptide (e.g. target polypeptide) to form a covalent (isopeptide) bond. The invention provides chimeric polypeptides comprising a self-processing module and their use in the production of polypeptides comprising an anhydride group. Methods of using the chimeric proteins to covalently link polypeptides and the products obtained from the methods are also provided, including their use in therapy and diagnosis. Related products, such as compositions comprising said chimeric proteins and polypeptides, nucleic acid molecules encoding said chimeric proteins, vectors comprising said nucleic acid molecules, and entities (e.g. host cells, exosomes, viruses, nanoparticles etc.) comprising said vectors, nucleic acid molecules and/or proteins and polypeptides also form aspects of the invention.

Covalent conjugation to proteins is desirable and can be advantageous over typical non-covalent coupling approaches. For example, decoration of a protein through a stable covalent bond can enhance long-term imaging, biomaterial strength, therapeutic/vaccine efficacy and diagnostic sensitivity. Much attention has focused on approaches involving the use of peptide tags able to react with catalytic protein partners, e.g. SpyTag, split inteins, sortase or OaAEP1, or on reactions through complementary click chemistry pairs. However, these approaches typically involve modification of both proteins and distinct strategies are required to conjugate moieties to unmodified endogenous proteins. Conjugation to unmodified endogenous proteins has greater relevance for therapeutic settings where it is desirable to minimise modifications to avoid unwanted immune responses. For this challenge, proximity-directed ligation has been an important approach, either using small molecules or protein binders. Small molecules with affinity for a target protein may be equipped with reactive functionalities, favouring covalent reaction with nearby nucleophiles in the binding site, e.g. cysteine residues. This approach has been successful for certain proteins, particularly those with deep and unique pockets facilitating specific ligand binding. However, attempts to generalize this approach to a wider range of protein targets have relied on post-translational modification or the use of unnatural amino acids, e.g. unnatural amino acids that have been genetically encoded. However, post-translational coupling of reactive groups or establishing unnatural amino acid incorporation in proteins is complex.

Other approaches for protein ligation have either used UV induction of highly reactive free radicals or weak electrophiles. UV-induced photocrosslinking is excellent for research applications but faces challenges for cellular use or use in living organisms because of the DNA-damaging phototoxicity and limited tissue penetration of UV light. The use of constitutive weak electrophiles for proximity ligation of proteins is a precarious balancing act between too low reactivity (leading to slow reaction) and too high reactivity (leading to non-specific coupling and spontaneous inactivation upon storage).

Thus, there remains a need for an approach for covalent targeting of proteins that can be applied generally to any endogenous protein.

The present inventors have established an approach for covalent targeting of endogenous proteins based on the standard genetic code, using chemistry that is inducible by mild, cell-friendly conditions. This is particularly advantageous as the expression of proteins based on the standard genetic code is generally easy, cheap and reliable. Moreover, the approach minimises additional sequences in the conjugation product. This facilitates the in vivo utility of the conjugation products, since even small peptide tags (e.g. 6 residues long) can induce immune responses.

In a representative embodiment, the invention utilises a self-processing module (SPM) that displays calcium-dependent autoproteolytic activity at an Asp-Pro bond to generate a reactive anhydride group on a polypeptide of interest. The reactive anhydride group is directed to react with an amine group, which may be present on the same protein, i.e. to produce an intramolecular isopeptide bond, or on another polypeptide (target polypeptide), i.e. to produce an intermolecular isopeptide bond. Thus, the approach may find utility in cyclizing polypeptides or in conjugating polypeptides. Moreover, the approach may be applied for specific protein targeting in vitro and on living cells.

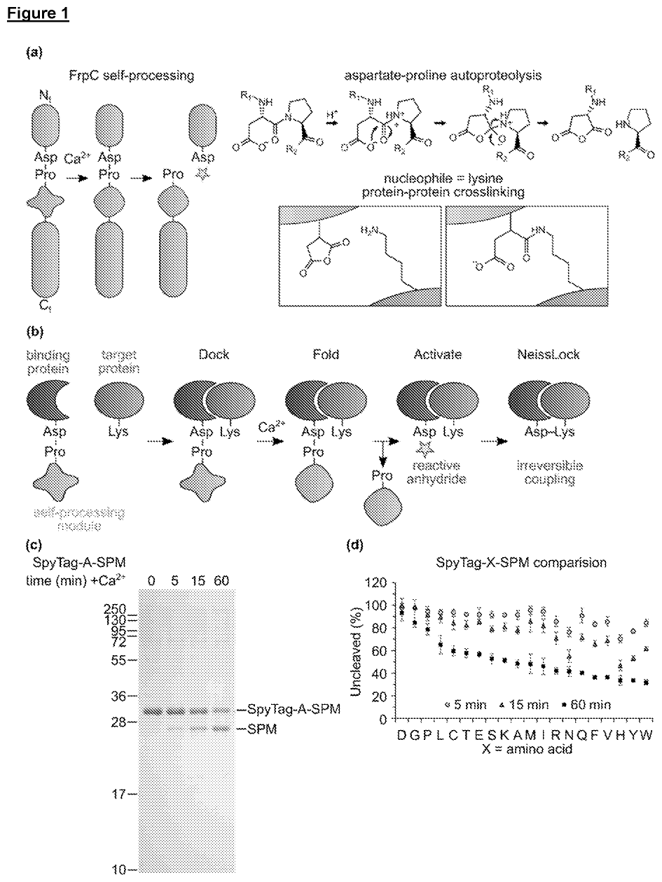

The polypeptide comprising the reactive anhydride may be directed to associate specifically with another polypeptide via a non-covalent interaction, i.e. the polypeptides to be conjugated may be selected on the basis that they are capable of interacting (e.g. binding) non-covalently. This non-covalent interaction promotes the proximity of the reactive anhydride and amine groups, thereby facilitating the formation of the isopeptide bond. Thus, the polypeptide on which the anhydride group is formed may be viewed as a “binding polypeptide” and the polypeptide with which it specifically interacts may be viewed as a “target polypeptide” (see FIG. 1b). Alternatively, the polypeptides may be viewed as a cognate pair that can be conjugated via an isopeptide bond when one of the polypeptides has been modified to comprise an anhydride group using a self-processing module.

As discussed in the Examples below, the approach has been exemplified primarily using the self-processing module from the FrpC protein of Neisseria meningitidis. Accordingly, the approach has been termed “NeissLock”. However, the inventors have also demonstrated that self-processing modules from other proteins also find utility in the claimed methods and uses.

Neisseria meningitidis FrpC is a secretory protein containing a self-processing module (SPM) which displays calcium-dependent autoproteolytic activity at an Asp-Pro bond. Moving from the low calcium environment inside the cell (Ca²⁺˜0.1 μM) to the extracellular medium (Ca²⁺ 1-2 mM) results in a calcium-dependent conformational change in SPM that mediates FrpC processing. While not wishing to be bound by theory, autoproteolysis is proposed to occur following protonation of Pro's main-chain nitrogen, leading to formation of an aspartic anhydride as an electrophile at the C-terminus of the proximal cleavage fragment, i.e. FrpC1-414 (FIG. 1a).

In Neisseria infection, this autoproteolysis appears to be involved in pathogen adhesion mediated by covalent conjugation of the proximal FrpC fragment to host cell proteins while bound to FrpD, a Neisserial outer membrane lipoprotein. Importantly, the FrpC region N-terminal to the Asp-Pro bond (FrpC1-413) is not required for autoproteolytic activity, so that SPM retains activity even when recombinantly fused to the C-terminus of various proteins.

As shown in the Examples, the inventors have determined that the residue preceding the Asp-Pro scissile bond was key to reactivity and may be used to design slow-acting or fast-acting covalent probes for NeissLock depending on the desired utility. The inventors surprisingly found that the NeissLock approach does not require precise apposition of the reacting nucleophile with the anhydride. A relatively large distance was predicted between the ε-amine of the nucleophilic Lys (K121 of ODC) and the last resolved residue of the binding protein, OAZ (where the anhydride is likely to be located) and yet efficient isopeptide bond formation was observed. Moreover, optimal activity at pH 6.5-7 was completely unexpected in view of the pKₐ of the ε-amine in lysine, which was predicted to allow reaction only at pH greater than 9 (where there is a substantial fraction of the amine in its deprotonated form). Moreover, the inventors have also determined that a range of nucleophiles on the target polypeptide (i.e. the α-amine or ε-amines) could rapidly react with the anhydride on the binding polypeptide, but reaction was blocked if the target polypeptide did not dock.

NeissLock therefore gives a system with intrinsic low reactivity (normal amino acid side-chains) until high reactivity is induced by the mild conditions of calcium concentrations typical for outside the cell. Then an anhydride is generated with high reactivity and can allow efficient coupling.

Calcium-inducibility means that the binding polypeptide may be incubated with the target polypeptide and excess binding polypeptide washed away before reactivity is induced, favouring specificity of coupling. However, even without washing away excess protein, the inventors found that lack of non-covalent interaction enabled minimal non-specific reaction with non-interacting proteins.

Thus, NeissLock facilitates the covalent conjugation of a broad range of protein assemblies, with both naturally existing and synthetic partners, under mild, cell-friendly conditions.

Thus, in one aspect, the present invention provides use of a chimeric protein to generate an anhydride group on a polypeptide, wherein the chimeric protein comprises:

(i) a domain comprising the polypeptide; and

(ii) a domain comprising a self-processing module that contains an N-terminal dipeptide of aspartate or glutamate and proline (D/E-P),

wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue in the self-processing module to release the polypeptide and generate the anhydride group on the aspartate or glutamate residue.

The reactive anhydride group generated on the polypeptide is used to direct the formation of a covalent bond. The anhydride group may react with various functional groups to form a covalent bond.

In preferred embodiments, the anhydride group reacts with an amine group to form an amide bond. In particularly preferred embodiments, the amine group is in an amino acid in a peptide or polypeptide (i.e. an α-amine or ε-amine). Thus, in some embodiments, the amide bond is a peptide bond or an isopeptide bond.

However, in some embodiments, the amine group is in an amino sugar or lipid. In some embodiments, the amino sugar or amine-containing lipid is covalently linked to a polypeptide. In some embodiments, the lipid forms part of a cell membrane. In some embodiments, the amino sugar forms part of an oligosaccharide or polysaccharide, i.e. the anhydride group reacts with an oligosaccharide or polysaccharide, e.g. an oligosaccharide or polysaccharide conjugated to a polypeptide. Thus, in some embodiments, the polypeptide comprising the anhydride group may be conjugated directly or indirectly to a second polypeptide (e.g. directly via an amide bond formed with an amino acid in the second polypeptide or indirectly via an amide bond with an amino sugar (e.g. in an oligosaccharide) conjugated to the second polypeptide).

In some embodiments, the amino sugar may be glucosamine, galactosamine or a conjugate thereof. In some embodiments, the oligosaccharide or polysaccharide is or comprises chitosan.

In some embodiments, the lipid is phosphatidylethanolamine, phosphatidylserine, sphingosine or a derivative thereof.

In some embodiments, the amide bond may form via a thioester bond. For instance, the anhydride group may react with a thiol group, e.g. in a cysteine residue (e.g. in a peptide or polypeptide) to form a thioester, which subsequently reacts with a nearby amine (e.g. an α-amine or ε-amine) to form an amide bond.

In some embodiments, the anhydride group may react with a hydroxyl group to form an ester. Thus, in some embodiments, the covalent bond is an ester bond. For instance, the hydroxyl group may be in the R-group of an amino acid, i.e. in serine, threonine or tyrosine. In some embodiments, the hydroxyl group may be in a sugar or lipid molecule. In some embodiments, the sugar or lipid is covalently linked (directly or indirectly) to a polypeptide. In some embodiments, the lipid forms part of a cell membrane. In some embodiments, the sugar forms part of an oligosaccharide or polysaccharide, i.e. the anhydride group reacts with an oligosaccharide or polysaccharide, e.g. an oligosaccharide or polysaccharide conjugated to a polypeptide.

Thus, in some preferred embodiments, the anhydride group reacts with a functional group, preferably an amine group, in a polypeptide to form an amide bond. In some embodiments, the amide bond is an intramolecular amide bond, i.e. the anhydride group reacts under suitable conditions with an amine group (an α-amine or ε-amine) within the same polypeptide, e.g. to cyclize the polypeptide.

Alternatively viewed, the invention provides a method of producing an anhydride group on a polypeptide comprising:

(a) providing a chimeric protein comprising:

(i) a domain comprising the polypeptide; and

(ii) a domain comprising a self-processing module that contains an N-terminal dipeptide of aspartate or glutamate and proline (D/E-P),

wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions;

(b) inducing the self-processing module to cleave the peptide bond between the proline residue and the aspartate or glutamate residue to release the polypeptide and generate the anhydride group on the aspartate or glutamate residue,

thereby producing a polypeptide comprising an anhydride group.

Thus, in some embodiments, the method or use may be viewed as enzymatically generating or producing an anhydride group on a polypeptide, wherein the anhydride group is for use in directing the formation of a covalent bond, e.g. an amide bond, e.g. an intramolecular amide bond within the polypeptide or an intermolecular amide bond between the polypeptide and another molecule, e.g. a second polypeptide.

Thus, in a further aspect, the invention provides a method of forming an intramolecular covalent bond (e.g. amide bond) in a polypeptide (e.g. a method of cyclizing a polypeptide) comprising:

(a) providing a chimeric protein comprising:

(i) a domain comprising the polypeptide; and

(ii) a domain comprising a self-processing module that contains an N-terminal dipeptide of aspartate or glutamate and proline (D/E-P),

wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions; and

(b) inducing the self-processing module to cleave the peptide bond between the proline residue and the aspartate or glutamate residue to release the polypeptide and generate an anhydride group on the aspartate or glutamate residue that reacts with a functional group (e.g. an amine group, hydroxyl group or thiol group) in the polypeptide to form a covalent bond (e.g. an amide bond, an ester bond or a thioester bond),

thereby forming an intramolecular covalent bond in the polypeptide (e.g. thereby cyclizing the polypeptide).

In some embodiments, it may be desirable to defer the formation of the isopeptide bond. For instance, the polypeptide comprising the anhydride group may be used as a reactant for subsequent conjugation to a target molecule. Thus, the method may comprise a step of isolating the polypeptide comprising an anhydride group and/or storing the polypeptide comprising an anhydride group under conditions in which the anhydride group is stable, e.g. in a non-aqueous solvent. Alternatively viewed, the polypeptide is stored under conditions that prevent hydrolysis or reaction of the anhydride group. Thus, the step of storing the polypeptide may involve adding a non-aqueous solvent (e.g. organic solvent, such as dimethylformamide (DMF) optionally containing a preservative such as an azide, such as sodium azide) to the polypeptide comprising an anhydride group (e.g. adding the non-aqueous solvent after step (b)). Additional steps may be used to stabilise the anhydride group, including maintaining the temperature of the solution comprising the polypeptide at about 10° C. or less, e.g. 9, 8, 7, 6, 5, 4° C. or less, such as about 0-10° C. or about 0-5° C., and/or at about 10° C. or less above the freezing point of the solution, e.g. −51° C. or less for DMF, e.g. −52, −53, −54 or less, such as about −56 to −61° C. for DMF.

Thus, in some embodiments, the invention provides the use of a chimeric protein as defined herein to produce a composition comprising a polypeptide comprising a stable anhydride group, e.g. wherein the composition contains a substance that prevents hydrolysis or reaction of the anhydride group and/or is stored under conditions that prevent hydrolysis or reaction of the anhydride group (e.g. temperature conditions as defined above). In some embodiments, the substance that prevents hydrolysis or reaction of the anhydride group is a non-aqueous solvent, i.e. present in an amount sufficient to prevent hydrolysis or reaction of the anhydride group.

In a further aspect, the invention provides a polypeptide comprising an anhydride group obtained by the method described above. A composition comprising a polypeptide comprising a stable anhydride group obtained by the method described above also forms an aspect of the invention.

However, as discussed in more detail below, in some embodiments, the polypeptide comprising the anhydride group is used as a reactant for subsequent conjugation to a target molecule immediately, e.g. within 20 minutes of the formation of the anhydride group, e.g. within 15, 10, 9, 8, 7, 6 or 5 minutes of the formation of the anhydride group. In this respect, formation of the anhydride group may be viewed as a suitable end-point of the reaction, e.g. wherein at least about 50%, preferably at least about 60% or 70% of the chimeric protein has been cleaved thereby generating the anhydride group. Additionally or alternatively, a suitable end-point may be within about 45 minutes of inducing the autoproteolytic reaction under suitable conditions as defined herein, e.g. within about 40, 35, 30, 25 or 20 minutes.

As shown in FIG. 1, cleavage of the chimeric protein in the N-terminal dipeptide (D/E-P) by the self-processing module results in the formation of the anhydride group on the aspartate or glutamate residue. Thus, following cleavage of the chimeric protein, the aspartate or glutamate residue is located at the C-terminus of the domain comprising the polypeptide (e.g. the binding polypeptide). In other words, cleavage of the chimeric protein results in the addition of an aspartate or glutamate residue (comprising an anhydride group) at the C-terminus of the domain comprising the polypeptide.

Thus, in a further embodiment the invention provides a polypeptide comprising an anhydride group on a C-terminal aspartate or glutamate residue, wherein the aspartate or glutamate residue in the polypeptide is not present at the equivalent position in the amino acid sequence of the corresponding endogenous polypeptide or portion thereof. Alternatively viewed, the aspartate or glutamate residue in the polypeptide does not correspond to an amino acid in the endogenous polypeptide or portion thereof.

Thus, in some embodiments, the polypeptide comprises an amino acid sequence that corresponds to the amino acid sequence of an endogenous polypeptide or a portion thereof except that the endogenous polypeptide or portion thereof does not contain an aspartate or glutamate residue at its C-terminus.

As discussed below, the chimeric protein may comprise a linker (also known as a spacer) domain between the domain comprising the polypeptide and the domain comprising the self-processing module. In these embodiments, the amino acid sequence of the polypeptide comprising the anhydride group will also differ from the amino acid sequence of its corresponding endogenous polypeptide or portion by virtue of the linker domain, i.e. polypeptide comprising the anhydride group will also contain the amino acids in the linker domain. In some embodiments, the polypeptide comprising the anhydride group may be provided in a composition and/or under conditions that prevents hydrolysis or reaction of the anhydride group (e.g. in a non-aqueous solvent and/or under temperature conditions as defined above).

The present invention also provides a polypeptide (e.g. a cyclized polypeptide) comprising an intramolecular covalent bond formed between an aspartate or glutamate residue and functional group in the polypeptide (e.g. an amine group such as on a lysine residue or at the N-terminus), wherein:

(i) the aspartate or glutamate residue in the polypeptide is not present in the amino acid sequence of the corresponding endogenous polypeptide or portion thereof; and

(ii) the functional group (e.g. amine group) in the polypeptide is present at an equivalent position (e.g. an equivalent position in the amino acid sequence) of the corresponding endogenous polypeptide or portion thereof.

The polypeptide comprising an intramolecular covalent (e.g. amide) bond (e.g. cyclized polypeptide) may be obtained by the method described above.

The term “cyclized” refers to the formation of ring structure within the polypeptide. For instance, a cyclized polypeptide may comprise a covalent bond between the C-terminal residue and an internal amino acid. In some less preferred embodiments, a cyclized polypeptide may be circularised comprising a covalent bond between the N-terminus and C-terminus. Cyclizing polypeptides has numerous potential advantages including: increasing protein activity (particularly enzyme activity) at higher temperature, increasing protein resilience to harsh conditions (e.g. after steam-treating of enzymes for animal feed) and inhibiting protease degradation.

The term “non-aqueous solvent” refers to any solvent that may be provided in a sufficient amount to prevent hydrolysis or reaction of the anhydride group. Selection of the solvent will depend on the properties of the polypeptide. In some preferred embodiments, the solvent is selected such that its addition to the polypeptide does not result in denaturation of the polypeptide or does not adversely affect the function of the polypeptide. In some embodiments, the non-aqueous solvent is an organic solvent, such as DMF, acetic acid, acetonitrile, N-methylformamide or N-methylacetamide. In some embodiments, the solvent may additional contain a preservative such as an azide, such as sodium azide or potassium azide.

In some embodiments, the covalent bond (e.g. amide bond, such as an isopeptide bond) formed by the reaction of the anhydride group and functional (e.g. amine) group is an intermolecular covalent (e.g. amide) bond, i.e. the anhydride group reacts under suitable conditions with functional group (e.g. an amine group, such as an α-amine or ε-amine) in another molecule, e.g. a different polypeptide, to conjugate the polypeptide comprising the anhydride group to the other molecule (e.g. polypeptide) via a covalent bond (e.g. an amide bond). Thus, in some embodiments, the polypeptide comprising the anhydride group may be termed a “first polypeptide” and the polypeptide comprising the functional group (e.g. amine group) that reacts to form the covalent (e.g. amide) bond may be termed a “second polypeptide”.

In preferred embodiments, the first polypeptide, in its unmodified form (i.e. not comprising a reactive anhydride group, i.e. in the chimeric protein) is capable of interacting non-covalently with the second polypeptide (i.e. binding selectively (e.g. specifically) and reversibly) such that, when the first polypeptide comprises the reactive anhydride group, the anhydride and functional (e.g. amine) group are brought into proximity facilitating the formation of the covalent (e.g. amide) bond. Thus, the polypeptide comprising the reactive anhydride group may be termed a “binding polypeptide” and the molecule (e.g. polypeptide) comprising the functional (e.g. amine) group may be termed a “target molecule” (e.g. “target polypeptide”). The binding polypeptide and target molecule (e.g. target polypeptide) may be viewed as a cognate pair.

Alternatively viewed, the domain comprising the polypeptide in the chimeric protein is capable of interacting non-covalently with the target molecule, e.g. second or target polypeptide, i.e. binding selectively and reversibly with the target molecule, e.g. second or target polypeptide. Thus, in some embodiments, the chimeric protein contains a domain comprising a binding polypeptide.

In some embodiments, the use of a chimeric protein to generate an anhydride group on a polypeptide may further comprise using the anhydride group on the polypeptide to conjugate the polypeptide to another molecule, e.g. a second polypeptide, via a covalent bond (e.g. an amide bond).

Accordingly in some embodiments, the invention provides the use of a chimeric protein to conjugate a first polypeptide to a second polypeptide via a covalent bond (e.g. an amide bond), wherein the chimeric protein comprises:

(i) a domain comprising the first polypeptide; and

(ii) a domain comprising a self-processing module that contains an N-terminal dipeptide of aspartate or glutamate and proline (D/E-P),

wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue in the self-processing module to release the first polypeptide and generate an anhydride group on the aspartate or glutamate residue at the C-terminus of the first polypeptide that reacts with a functional group (e.g. an amine group) on the second polypeptide to form the covalent bond (e.g. amide bond).

As noted above, the first polypeptide, in its unmodified form (i.e. not comprising a reactive anhydride group, i.e. in the form of the chimeric protein), is capable of interacting non-covalently with the second polypeptide, i.e. the first and second polypeptides are capable of binding selectively and reversibly. Once the first polypeptide is modified to comprise an anhydride group, the non-covalent interaction with the second polypeptide promotes the formation of the covalent bond (e.g. amide bond), i.e. the non-covalent interaction promotes the proximity-directed ligation of the polypeptides via reaction of the anhydride group and functional group (e.g. amine group) to form a covalent bond (e.g. an amide bond). Alternatively viewed, the first and second polypeptides may be viewed as a cognate pair that can be conjugated via a covalent bond (e.g. an amide bond) when one of the polypeptides has been modified to comprise an anhydride group using a self-processing module.

Thus, the “chimeric protein” may be viewed as a “covalent probe” or “probe” that is capable of mediating the covalent conjugation of a polypeptide to a target molecule (e.g. polypeptide) via a covalent bond (e.g. an amide bond).

In a further embodiment, the invention provides a method of conjugating a first polypeptide to a second polypeptide via a covalent bond (e.g. an amide bond) comprising:

(a) providing a chimeric protein comprising:

(i) a domain comprising the first polypeptide; and

(ii) a domain comprising a self-processing module that contains an N-terminal dipeptide of aspartate or glutamate and proline (D/E-P),

wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions;

(b) contacting the chimeric protein of (a) with the second polypeptide, wherein the second polypeptide binds non-covalently to (i);

(c) inducing the self-processing module to cleave the peptide bond between the proline residue and the aspartate or glutamate residue to release the first polypeptide and generate an anhydride group on the aspartate or glutamate residue that reacts with a functional group (e.g. an amine group) on the second polypeptide to form an isopeptide bond, thereby conjugating the first and second polypeptides.

In a further embodiment, the invention provides a product comprising a first polypeptide conjugated to a second polypeptide via a covalent bond (e.g. an amide bond) between an aspartate or glutamate residue in the first polypeptide and a functional group (e.g. an amine group such as in a lysine residue) in the second polypeptide, wherein:

(i) the aspartate or glutamate residue in the first polypeptide is not present at the equivalent position in the amino acid sequence of the corresponding endogenous polypeptide or portion thereof; and

(ii) the functional group (e.g. amine group) in the second polypeptide is present at the equivalent position (e.g. equivalent position in the amino acid sequence) of the corresponding endogenous polypeptide.

In some embodiments, the first polypeptide comprises an amino acid sequence that corresponds to the amino acid sequence of an endogenous polypeptide or a portion thereof except that the endogenous polypeptide or portion thereof does not contain an aspartate or glutamate residue at its C-terminus, and the second polypeptide comprises an amino acid sequence that corresponds to the amino acid sequence of an endogenous polypeptide or a portion thereof which contains a functional group (e.g. an amine group such as in a lysine residue) at an equivalent position to the functional group (e.g. amine group, e.g. lysine residue) in the second polypeptide.

The product comprising a first polypeptide conjugated to a second polypeptide via a covalent bond (e.g. an amide bond) may obtained by the method described above and this forms a further aspect of the invention.

The term “chimeric protein” refers to a protein comprising two or more polypeptides (e.g. proteins or protein subunits (also known as protein domains)) linked together (end-to-end), wherein the polypeptides are not found linked together in nature. Thus, a chimeric protein is not a native protein. Accordingly, a chimeric protein may comprise polypeptides that are derived from different sources, or polypeptides derived from the same source, but arranged in a manner different than that found in nature. The two or more polypeptides may be joined together by one or more peptide linkers, e.g. polypeptide-peptide linker-polypeptide. Advantageously, chimeric proteins may be created through the joining of two or more nucleic acids (e.g. genes) that originally coded for separate polypeptides. Thus, a chimeric protein may alternatively be termed a “fusion protein”.

As used herein, a chimeric protein refers to a protein comprising (i) a domain comprising the polypeptide on which it is desirable to generate an anhydride group; and (ii) a domain comprising a self-processing module, wherein (i) and (ii) are linked by a peptide bond. In some embodiments, the chimeric protein comprises (i) a domain comprising the polypeptide on which it is desirable to generate an anhydride group; (ii) a peptide linker; and (iii) a domain comprising a self-processing module, wherein (i) and (ii), and (ii) and (iii) are each linked by a peptide bond. The order of domains (i)-(iii) in the chimeric protein is N-terminal to C-terminal. Thus, when a peptide linker is present in the chimeric protein, the domain comprising the polypeptide on which it is desirable to generate an anhydride group; and the domain comprising a self-processing module are indirectly linked by a peptide bond, i.e. each domain is directly linked to the peptide linker via a peptide bond.

A “domain” refers to a discrete, continuous part or subsequence of a polypeptide that can be a potentially independent, stable folding unit and may be associated with one or more functions. Thus, in the context of the chimeric protein of the invention, a domain may contain the specified components, e.g. the first polypeptide (e.g. binding polypeptide) or self-processing module, and may contain other components. Thus, a domain may be viewed as a “region” of the chimeric protein containing one or more polypeptide elements. The terms “domain” and “region” may be used interchangeably herein. In some embodiments, the domains of the chimeric protein consist of the specified components, particularly the peptide linker and self-processing module. Thus, in some embodiments, only the domain comprising the polypeptide on which it is desirable to generate an anhydride group may contain additional polypeptide sequences. However, in some embodiments, the domain comprising the self-processing module may advantageously contain an affinity tag, e.g. His-tag, C-tag, FLAG-tag, SpyTag etc, e.g. it may consist of the self-processing module and an affinity tag.

For instance, where the target molecule does not contain a naturally-occurring binding partner (e.g. polypeptide) or it is desirable to conjugate the target molecule to a polypeptide that does not bind to the target molecule, it may be advantageous to generate a fusion protein containing a polypeptide that binds to the target molecule linked (e.g. directly via a peptide bond or indirectly via a peptide linker) to the polypeptide to be conjugated to the target molecule. Alternatively viewed, domain (i) may contain a polypeptide capable of binding non-covalently to the target molecule and a polypeptide to be conjugated to the target molecule.

A “self-processing module” or “SPM” refers to a functional domain of a polypeptide that displays calcium-dependent autoproteolytic activity at an Asp-Pro (D-P) or Glu-Pro (E-P) bond that results in the cleavage of a polypeptide comprising the SPM, wherein the N-terminal cleavage product comprises a reactive anhydride group on the Asp or Glu at the C-terminus. Any suitable SPM may be used in the chimeric protein of the present invention.

In some embodiments, the SPM is from a bacterial protein, e.g. a secretory protein, such as from Alysiella sp., Kingella sp. or Neisseria sp., preferably a secretory protein from Alysiella filiformis, Kingella negevensis or Neisseria meningitidis. Thus, in some embodiments, the SPM is derived from the FrpA or FrpC protein of Neisseria meningitidis, i.e. the SPM is the SPM from FrpA or FrpC (preferably FrpA) of Neisseria meningitidis or a functional variant, portion and/or derivative thereof. Suitable SPMs may readily be obtained through homology-based searching of protein databases using the polypeptide sequences exemplified herein and search tools well-known in the art and described herein (e.g. FASTA, BLAST).

As shown in the Examples, the inventors have determined that self-processing modules with divergent sequences may find utility in the chimeric protein of the invention. For instance, the SPM from the bifunctional haemolysin/adenylate cyclase precursor protein from Kingella negevensis (SEQ ID NO: 4), which shows just 60.41% sequence identity to the SPM from FrpC protein from Neisseria meningitidis (SEQ ID NO: 2).

Thus, in some embodiments, the SPM or functional variant or derivative thereof, comprises an amino acid sequence with at least 60% sequence identity to a sequence as set forth in any one of SEQ ID NOs: 1-4. In some embodiments, the functional variant or derivative is a hyperactive variant or derivative, i.e. a variant or derivative with increased autoproteolytic activity relative to the naturally-occurring protein.

Preferably said polypeptide sequence is at least 65, 70, 75, 80, 85, 90, 91, 92, 93, 94, 95, 96, 97, 98 or 99% identical to the sequence to which it is compared.

Sequence identity of polypeptide molecules may be determined by, e.g. using the SWISS-PROT protein sequence databank using FASTA pep-cmp with a variable pamfactor, and gap creation penalty set at 12.0 and gap extension penalty set at 4.0, and a window of 2 amino acids. Preferably said comparison is made over the full length of the sequence, but may be made over a smaller window of comparison, e.g. less than 200, 100 or 50 contiguous amino acids.

Preferably such sequence identity related polypeptides are functionally equivalent to one of the polypeptides set forth in SEQ ID NOs: 1-4, preferably functionally equivalent to polypeptides set forth in SEQ ID NOs: 1 or 2. As such, the polypeptides with a sequence as set forth in SEQ ID NOs: 1-4 may be modified without affecting the sequence of the polypeptide.

Modifications that do not affect the sequence of the polypeptide include, e.g. chemical modification, including by deglycosylation or glycosylation. Such polypeptides may be prepared by post-synthesis/isolation modification of the polypeptide without affecting functionality, e.g. glycosylation, methylation etc. of particular residues.

As referred to herein, to achieve “functional equivalence” the polypeptide may show some increased or reduced autoproteolytic activity (e.g. cleavage of the D-P or E-P peptide bond) relative to the parent molecule (i.e. the molecule from which it was derived, e.g. by amino acid substitution), but preferably is as efficient or is more efficient. Thus, functional equivalence relates to a polypeptide which has autoproteolytic activity capable of cleaving of the D-P or E-P peptide bond under suitable conditions, e.g. in the presence of calcium ions. This may be tested by comparison of the autoproteolytic activity of the derivative polypeptide relative to the polypeptide from which it is derived in a quantitative manner. The derivative is preferably at least 30, 50, 70 or 90% as effective as the parent polypeptide in the methods of the invention. As noted above, in some preferred embodiments, the polypeptide is hyperactive relative to the parent polypeptide exemplified above, i.e. is at least about 110, 120, 130, 140, 150, 200, 250 or 300% as effective as the parent polypeptide in the methods of the invention.

Functionally-equivalent proteins, which are related to or derived from the naturally-occurring proteins exemplified herein, may be obtained by modifying the native amino acid sequence by single or multiple amino acid substitution, addition and/or deletion (providing they satisfy the above-mentioned sequence identity requirements), but without destroying the molecule's function. Preferably the modified sequence has less than 50 substitutions, additions or deletions, e.g. less than 40, 30, 25, 20, 15, 10, 5, 4, 3 or 2 such modifications, relative to the native sequence. Such proteins are encoded by “functionally-equivalent nucleic acid molecules” which are generated by appropriate substitution, addition and/or deletion of one or more nucleotides.

As described in the Examples, C-terminal truncated forms of the SPM retain autoproteolytic activity. Thus, the polypeptides exemplified herein (SEQ ID NOs: 1-4) may be truncated by up to 67 amino acids at the C-terminus (e.g. by about 1, 2, 3, 4, 5, 10, 20, 30, 40, 50, 60 or 65 amino acids). Thus, the term variant as used herein includes truncation variants of the exemplified polypeptides. Alternatively, viewed, the invention may be seen to provide portions of the exemplified polypeptides, wherein said portions comprise an amino acid sequence as set forth in any one of SEQ ID NOs: 5-8 or a variant or derivative thereof, as discussed above.

As referred to herein a “portion” comprises at least an amino acid sequence as set forth in one of SEQ ID NOs: 5-8, i.e. at least 175, 180, 190, 200, 210, 220, 230, 240 or more amino acids of one of SEQ ID NOs: 1-4 (the sequence from which it is derived) containing an amino acid sequence as set forth in one of SEQ ID NOs: 5-8. Thus, said portion is obtained from the N-terminal portion of the sequence, i.e. the portion comprises the N-terminal sequence of one of SEQ ID NOs: 1-4; it is a C-terminal truncation. Notably, “portions” as described herein are polypeptides of the invention and therefore satisfy the identity conditions (relative to a comparable region) and functional equivalence conditions mentioned herein.

Thus, in some embodiments, the chimeric protein, e.g. for use in the methods and uses of the invention, comprises N-terminus to C-terminus:

(i) a domain comprising a polypeptide; and

(ii) a domain comprising a self-processing module comprising:

(1) an amino acid sequence as set forth in any one of SEQ ID NOs: 1-4;

(2) a portion of (1) comprising an amino acid sequence as set forth in any one of SEQ ID NOs: 5-8;

(3) an amino acid sequence with at least 60% sequence identity to an amino acid sequence as set forth in any one of SEQ ID NOs: 1-4; or

(4) a portion of (3) comprising an amino acid sequence with at least 60% sequence identity to an amino acid sequence as set forth in any one of SEQ ID NOs: 5-8,

wherein the first (N-terminal) amino acid of the domain comprising a self-processing module is an aspartate or glutamate and the second amino acid of the domain comprising a self-processing module is proline;

and wherein the self-processing module cleaves the peptide bond between the first and second amino acids of the domain comprising a self-processing module under suitable conditions.

In some embodiments the self-processing module comprises:

(1) an amino acid sequence as set forth in SEQ ID NO: 1;

(2) a portion of (1) comprising an amino acid sequence as set forth in SEQ ID NO: 5;

(3) an amino acid sequence with at least 99% sequence identity to an amino acid sequence as set forth in SEQ ID NO: 1; or

(4) a portion of (3) comprising an amino acid sequence with at least 99% sequence identity to an amino acid sequence as set forth in SEQ ID NO: 5,

wherein the amino acid sequence comprises aspartate or glutamate at position 1 and proline at position 2;

and wherein the self-processing module cleaves the peptide bond between the first and second amino acids of the domain comprising a self-processing module under suitable conditions.

In some embodiments, the self-processing module comprises:

(1) an amino acid sequence as set forth in SEQ ID NO: 1;

(2) a portion of (1) comprising an amino acid sequence as set forth in SEQ ID NO: 5;

(3) an amino acid sequence with at least 80% sequence identity to an amino acid sequence as set forth in SEQ ID NO: 1 or 2; or

(4) a portion of (3) comprising an amino acid sequence with at least 80% sequence identity to an amino acid sequence as set forth in SEQ ID NO: 5 or 6,

wherein the amino acid sequence comprises aspartate or glutamate at position 1, proline at position 2 and one or more of the following:
1) alanine at position 17;
2) alanine at position 23;
3) arginine at position 28;
4) glutamine at position 30;

and wherein the self-processing module cleaves the peptide bond between the first and second amino acids of the domain comprising a self-processing module under suitable conditions.

In some embodiments, the self-processing module contains two of the amino acid residues specified in 1)-4) above, i.e. 1) and 2), 1) and 3), 1) and 4), 2) and 3), 2) and 4) or 3) and 4). In some embodiments, the self-processing module contains three of the amino acid residues specified in 1)-4) above, i.e. 1), 2) and 3), 1), 3) and 4), 1), 2) and 4) or 2), 3) and 4). In some embodiments, the self-processing module contains all of the amino acid residues specified in 1)-4) above.

The numbering refers to the numbering of SEQ ID NOs: 1 and 2 and encompasses equivalent positions, which can be deduced by lining up the sequence of the homologue (mutant, variant or derivative) polypeptide and the sequence of SEQ ID NO: 1 or 2 based on the homology or identity between the sequences, for example using a BLAST algorithm.

In some embodiments, domain (ii) consists of the self-processing module defined above.

The inventors have determined that the amino acid residue preceding the Asp-Pro or Glu-Pro scissile bond in the self-processing module has an effect on the reactivity of the SPM. Thus, in some embodiments, the polypeptide in domain (i) of the chimeric protein may have a C-terminal amino acid that facilitates the desired reactivity of the SPM, e.g. an amino acid selected from R, N, Q, F, V, H, Y or W (preferably H, Y or W) where high reactivity is required. However, when the polypeptide in domain (i) of the chimeric protein does not have a C-terminal amino acid that facilitates the desired reactivity of the SPM, it may be useful to include a peptide linker between the C-terminal amino acid of domain (i) and the aspartate or glutamate of the SPM, such that the amino acid residue preceding the Asp-Pro or Glu-Pro scissile bond promotes the desired reactivity.

The inventors have also determined that increasing the length of the linker (i.e. including a spacer sequence) may also improve the reactivity of the SPM. Thus, in some embodiments, the peptide linker may contain more than one amino acid, e.g. 2, 3, 4, 5 or more amino acids, e.g. 2-25, 2-20, 2-15 or 2-10 amino acids, preferably 1-5.

The spacer sequence may be of variable length and/or sequence, for example it may have 2-20, 1-15, 1-12, 1-10, 1-8, or 1-6 residues, e.g. 6, 7, 8, 9, 10 or more residues. By way of representative example the spacer sequence, if present, may have 1-15, 1-12, 1-10, 1-8 or 1-6 residues etc. The residues may for example be any amino acid, e.g. a neutral amino acid, or an aliphatic amino acid, or alternatively they may be hydrophobic, or polar or charged or structure-forming, e.g. proline. In some preferred embodiments, the linker is a serine and/or glycine-rich sequence.

Accordingly in some embodiments, the chimeric protein comprises N-terminus to C-terminus:

(i) a domain comprising a polypeptide;

(ii) a domain comprising a linker; and

(iii) a domain comprising a self-processing module as defined above.

In some embodiments, the linker consists of a single amino acid selected based on the level of reactivity required. Where it is desirable to generate the anhydride group on the polypeptide slowly, the linker may be selected from D, G, P. In some embodiments, the linker is not D, G or P. Where it is desirable to generate the anhydride group on the polypeptide with intermediate rate, the linker may be selected from L, C, T, E, S, K, A, M or I. Where it is desirable to generate the anhydride group on the polypeptide quickly, the linker may be selected from R, N, Q, F, V, H, Y or W, preferably V, H, Y or W.

Alternatively viewed, the polypeptide in domain (i) of the chimeric protein may have a C-terminal amino acid selected from D, G, P, L, C, T, E, S, K, A, M or I, preferably L, C, T, E, S, K, A, M or I, or a C-terminal amino acid selected from R, N, Q, F, V, H, Y or W, preferably V, H, Y or W.

In some embodiments, the chimeric protein comprises linker with the motif X₁X₂X₃, wherein:

(a) X₁ and X₂ are independently selected from any amino acid, preferably G and S (e.g. GS, SG or GG); and

(b) X₃ is selected from R, N, Q, F, V, H, Y or W, preferably V, H, Y or W (e.g. H, Y or W, or H or W).

In some embodiments, the amino acid preceding the Asp-Pro or Glu-Pro scissile bond (e.g. X₃) is not Y. For instance, in some embodiments, when the SPM is one of SEQ ID NOs: 1, 2 or 4 (particularly SEQ ID NO: 2) or a variant or portion thereof as defined above, the amino acid preceding the Asp-Pro or Glu-Pro scissile bond is not Y. In some embodiments, when the SPM is SEQ ID NO: 3 or a variant or portion thereof as defined above, the amino acid preceding the Asp-Pro or Glu-Pro scissile bond (e.g. X₃) is not V.

A chimeric protein comprising a linker as defined above forms a further aspect of the invention. Similarly, products of the methods described above may also contain a linker as defined above, as the linker will be contained in the N-terminal cleavage product of the autoproteolytic reaction.

The SPM polypeptides exemplified herein display calcium-dependent autoproteolytic activity at an Asp-Pro or Glu-Pro bond, e.g. autoproteolytic activity is induced or promoted by the present of Ca²⁺ at a concentration of at least about 0.1 mM.

Thus, conditions that are suitable to induce the cleavage of the Asp-Pro or Glu-Pro bond in the SPM include the presence of Ca²⁺ at a concentration of at least about 0.1 mM, e.g. about 0.25, 0.5, 1.0. 1.5, 2, 2.5, 3, 3.5, 4, 5, 6, 7, 8, 9, 10 mM or more. Alternatively viewed, the self-processing module cleaves the peptide bond between the proline residue and the aspartate or glutamate residue in the chimeric protein in the presence of Ca²⁺ at a concentration of at least about 0.1 mM, e.g. about 0.25, 0.5, 1.0, 1.5, 2, 2.5, 3, 3.5, 4, 5, 6, 7, 8, 9, 10 mM or more.

Thus, in some embodiments, the step of inducing the self-processing module to cleave the peptide bond between the proline residue and the aspartate or glutamate residue in the chimeric protein to release the polypeptide and generate an anhydride group on the aspartate or glutamate residue comprises contacting the chimeric protein with Ca²⁺ at a concentration of at least about 0.1 mM, e.g. about 0.25, 0.5, 1.0, 1.5, 2, 2.5, 3, 3.5, 4, 5, 6, 7, 8, 9, 10 mM or more. For instance, the step may comprise adding a buffer comprising Ca²⁺ to a solution comprising the chimeric protein such that the final concentration of Ca²⁺ is at least about 0.1 mM, e.g. about 0.25, 0.5, 1.0, 1.5, 2, 2.5, 3, 3.5, 4, 5, 6, 7, 8, 9, 10 mM or more. The Ca²⁺ may be provided in any suitable form, such as a calcium chloride solution.

In some embodiments, inducing the self-processing module to cleave the peptide bond between the proline residue and the aspartate or glutamate residue in the chimeric protein to release the polypeptide and generate an anhydride group on the aspartate or glutamate residue comprises introducing the chimeric protein to an environment with Ca²⁺ at a concentration of at least about 0.1 mM, e.g. about 0.25, 0.5, 1.0, 1.5, 2, 2.5, 3, 3.5, 4, 5, 6, 7, 8, 9, 10 mM or more. For instance, introducing (e.g. exposing) the chimeric protein to an in vivo environment comprising the specified calcium concentration. For instance, the chimeric protein may be introduced to an in vivo environment by injection into a body or tissue as described below or by expression within a cell, e.g. an in vivo translated protein (produced from an introduced nucleic acid molecule encoding the protein) may be translocated to an intracellular compartment with the required calcium concentration, e.g. endoplasmic reticulum, or outside the cell. Thus, in some embodiments, the chimeric protein may comprise a signal peptide that functions to translocate the protein to an intracellular compartment or into the extracellular matrix (i.e. targets the chimeric protein or the product of the invention for secretion).

It is evident from the Examples below that the chimeric protein of the invention (i.e. the SPM of the chimeric protein) is active under a range of conditions. For instance, in HEPES buffer at a pH of 6.0-9.0, e.g. 6.0-8.5, such as about 6.5-7.0, over a range of temperatures, e.g. 0-40° C., such as 5-39, 10-38, 15-37° C., e.g. 1, 2, 3, 4, 5, 10, 12, 15, 18, 20, 22, 25, 27, 29, 31, 33, 35 or 37° C., preferably about 37° C. The chimeric protein is functional in the presence of extracellular concentrations of NaCl, e.g. about 150 mM NaCl or less. However, in some embodiments, it may be preferable to induce autoproteolytic activity in the absence of NaCl. The skilled person would readily be able to determine other suitable conditions.

Thus, in some embodiments, conditions that are suitable to induce or promote the autoproteolytic activity of the SPM includes any conditions in which the addition of at least about 0.1 mM Ca²⁺ to the chimeric protein of the invention results in the cleavage of the Asp-Pro or Glu-Pro bond and the formation of an anhydride group on the Asp or Glu residue. For instance, addition of buffer comprising Ca²⁺ to said chimeric protein in buffered conditions, e.g. in a buffered solution or on a solid phase (e.g. column) that has been equilibrated with a buffer, such as HEPES buffer, such that the final concentration of Ca²⁺ is at least about 0.1 mM. The step of inducing autoproteolysis may be at any suitable pH, such as about pH 6.0-9.0, e.g. about pH 6.0, 6.2, 6.4, 6.6, 6.8, 7.0, 7.2, or 7.4. Additionally or alternatively, the step of inducing autoproteolysis may be at any suitable temperature, such as about 0-40° C., e.g. about 5-40, 10-39, 20-38 or 25-37° C., e.g. about 20, 25, 30, 35 or 37° C., preferably about 37° C. In some embodiments, the step of contacting may be in the absence of NaCl. In some embodiments, inducing autoproteolysis may be in the presence of a reducing agent, such as (tris(2-carboxyethyl)phosphine) (TCEP, e.g. TCEP-HCl). In some embodiments, the reducing agent, e.g. TCEP, is present in the reaction at a concentration of at least about 0.5 mM, e.g. about 0.5-5.0 mM, such as about 2.0 mM.

The term “generate an anhydride group on the aspartate or glutamate residue” refers to the formation of the anhydride group on the aspartate or glutamate residue of the N-terminal dipeptide that is cleaved by the SPM. The reaction mechanism is shown in FIG. 1a. Thus, the anhydride group is generated on the aspartate or glutamate group by inducing autoproteolysis as described above.

The terms “inducing autoproteolysis” and “inducing the self-processing module to cleave the peptide bond between the proline residue and the aspartate or glutamate residue in the chimeric protein” may be viewed as activating the SPM.

The terms “N-terminal”, “N-terminus”, “C-terminal” and “C-terminus” are used herein to refer to the position of amino acid residues within the polypeptides and proteins (e.g. chimeric proteins), and domains thereof, described herein. For example, the reference to N-terminal amino acid does not necessarily mean that the amino acid is at the amino terminus of the polypeptide or protein (i.e. comprising an α-amine group and linked only to one other amino acid). An N-terminal amino acid or peptide may refer to the internal position of the amino acid or peptide within the polypeptide or domain, i.e. an amino acid or peptide located at the N-terminal end of a domain which is coupled via a peptide bond to the C-terminal end of the “upstream” domain. Similarly, a C-terminal amino acid or peptide may refer to an amino acid or peptide located at the C-terminal end of a domain that is coupled via a peptide bond to the N-terminal end of the “downstream” domain. However, in some embodiments, the terms N-terminus and C-terminus refer to the end residues of the polypeptides described herein, i.e. the amino acids comprising the terminal amine and carboxyl groups. The meaning of these terms will be clear to the skilled person based on the context of their use.

Thus, polypeptides that form the domains of the chimeric protein of the invention may be isolated, purified, recombinant or synthesized polypeptides. The terms “peptide”, “polypeptide” and “protein” are used herein interchangeably herein and these terms includes any amino acid sequence comprising at least about 4 consecutive amino acids, such as at least about 5, 6, 7, 8, 9, 10, 12, 15, 20, 25 or 30 amino acids. In some embodiments, the term polypeptide refers to any amino acid sequence comprising at least about 40 consecutive amino acid residues, e.g. at least 50, 60, 70, 80, 90, 100, 150 amino acids, such as 40-1000, 50-900, 60-800, 70-700, 80-600, 90-500, 100-400 amino acids. There is no standard definition regarding the size boundaries between what is meant by peptide and polypeptide, but typically a peptide may be viewed as comprising between 2-39 amino acids. Thus, in some embodiments, domain (i) of the chimeric protein may be viewed as containing a peptide. Similarly, in some embodiments, the target polypeptide may be viewed as a peptide. Thus, in some embodiments, the methods and uses described herein may be viewed as conjugating two peptides or a peptide and a polypeptide.

It will be evident that any polypeptides may be used in domain (i) of the chimeric protein of the invention. Thus, domain (i) of the chimeric protein may contain any desired polypeptide. In other words, the invention may utilise any polypeptide in which it is desired to introduce an intramolecular covalent bond (e.g. to cyclize and/or stabilise the polypeptide). Similarly, where it is desirable to conjugate two polypeptides via an isopeptide bond, either of the polypeptides (e.g. polypeptides of a cognate pair) may be used in domain (i) of the chimeric polypeptide.

While the specific formation of the covalent bond (e.g. amide bond) between polypeptides is a proximity-based reaction promoted by the non-covalent (e.g. reversible) binding of the polypeptides, it will be evident that one or both of the polypeptides for conjugation may be modified to include domains that facilitate the non-covalent binding of the polypeptides. Thus, when the polypeptides selected for conjugation do not naturally interact (are not capable of binding to each other specifically, non-covalently and reversibly, e.g. are not a natural cognate pair), one of the polypeptides may be provided with a binding domain to enable non-covalent binding of the polypeptides, i.e. to promote the proximity-directed reaction of the anhydride and amine groups. In some embodiments, a suitable binding domain may be selected by screening a library of chimeric proteins containing variant binding domains (e.g. antibody-like domains) in domain (i) against the target polypeptide, e.g. selecting the binding domain that is conjugated to the target polypeptide. In some embodiments, the binding domain may be derived from a polypeptide that is known to interact with the target polypeptide.

In embodiments where one of the polypeptides is provided with a binding domain, it is preferred that the binding domain forms part of domain (i) of the chimeric protein. However, in preferred embodiments, the polypeptides to be conjugated are capable of binding to each other specifically and non-covalently without the addition of a heterologous binding domain, i.e. the polypeptides are a natural or native cognate pair.

As the binding polypeptide and/or the target polypeptide may be modified to promote a specific and reversible non-covalent interaction, it will be evident that any polypeptide containing a suitable functional group (e.g. amine group) may be used as the target polypeptide. Thus, when selecting polypeptides for use in the methods and uses of the invention, it may be advantageous to select the target protein and subsequently identify and/or manufacture a suitable binding polypeptide.

The term “cognate” refers to components that function or specifically interact together. Thus, in the context of the present invention, a cognate pair refers to a binding polypeptide and target molecule (target polypeptide) that bind non-covalently to form a complex (e.g. a polypeptide complex).

The term “binds selectively” refers to the ability of the binding polypeptide to bind non-covalently (e.g. by van der Waals forces and/or ionic interactions and/or hydrogen-bonding) to its target polypeptide (i.e. cognate polypeptide) with greater affinity and/or specificity than to other components in the sample in which the target polypeptide is present. Thus, the binding polypeptide (e.g. in the form of the chimeric protein comprising the binding polypeptide) may alternatively be viewed as binding specifically and reversibly to the target polypeptide under suitable conditions.

Binding to the target polypeptide may be distinguished from binding to other molecules (e.g. peptides or polypeptides) present in the sample, i.e. non-cognate molecules. The binding polypeptide either binds less efficiently to other molecules (e.g. peptides or polypeptides) present in the sample or does so negligibly or non-detectably that any such non-specific binding, if it occurs, readily may be distinguished from binding to the target polypeptide.

In particular, if the binding polypeptide binds to molecules other than the target polypeptide, such binding must be transient and the binding affinity must be less than the binding affinity of the binding polypeptide for the target polypeptide. Thus, the binding affinity of the binding polypeptide for the target polypeptide should be at least an order of magnitude more than the other molecules (i.e. non-cognate molecules) present in the sample. Preferably, the binding affinity of the binding polypeptide for the target polypeptide should be at least 2, 3, 4, 5, or 6 orders of magnitude more than the binding affinity for non-cognate molecules (e.g. peptides or polypeptides).

Thus, selective or specific binding refers to affinity of the binding polypeptide for its target polypeptide where the dissociation constant (K_{d}) of the binding polypeptide for the target polypeptide is less than about 10⁻³ M. In a preferred embodiment, the dissociation constant of the binding polypeptide for the target polypeptide is less than about 10⁻⁴ M, 10⁻⁵ M, 10⁻⁶ M, 10⁻⁷ M, 10⁻⁸ M or 10⁻⁹ M. Alternatively viewed, the dissociation constant (K_{d}) of the binding polypeptide for the non-target molecules (e.g. polypeptides) is more than about 10⁻³ M, e.g. 0.01 M, 0.1 M.

Suitable conditions for the selective or specific binding of the binding polypeptide to its target polypeptide will be dependent on the structures and functions of the polypeptides. Selection of suitable conditions is within the purview of the skilled person.

The term “reversible” or “binds reversibly” refers to a non-covalent interaction between the binding polypeptide and the target polypeptide, e.g. an interaction that can be disrupted without cleavage of a covalent bond.

The term “binding domain” refers to a polypeptide domain capable of binding selectively to its binding partner, which may be a polypeptide or non-polypeptide entity (e.g. a sugar, oligosaccharide, polysaccharide or lipid as described above). For instance, domain (i) of the chimeric protein may comprise a binding domain linked to the desired polypeptide (the polypeptide to be conjugated to the target polypeptide) to provide the “binding polypeptide”. The binding domain may bind selectively to an epitope (domain) in the target polypeptide (e.g. an amino acid domain). In some embodiments, the binding domain may be a portion of a polypeptide that naturally interacts with the target polypeptide (i.e. a portion that is sufficient to mediate a specific interaction, i.e. non-covalent binding). Alternatively, the binding domain may be a synthetic or manufactured interaction partner, e.g. an antibody fragment such as an scFv. In some embodiments, the binding domain may be a polypeptide, e.g. streptavidin, maltose binding domain or an antibody (e.g. scFv), that interacts with a moiety that has been introduced to the target polypeptide, e.g. biotin, maltose or a hapten.

In some embodiments, the chimeric protein and target molecule (e.g. target polypeptide) bind indirectly. In other words, the non-covalent interaction between the chimeric protein (i.e. domain (i) of the chimeric protein) and the target molecule (e.g. target polypeptide) is mediated via one or more other molecules. For instance, the chimeric protein binds non-covalently to a molecule (e.g. antibody) that binds non-covalently to the target molecule (e.g. target polypeptide). Thus, the molecule that mediates the interaction between chimeric protein and the target molecule contains a first region (e.g. epitope) that binds to domain (i) of the chimeric protein and a second region (e.g. epitope) that binds to the target molecule.

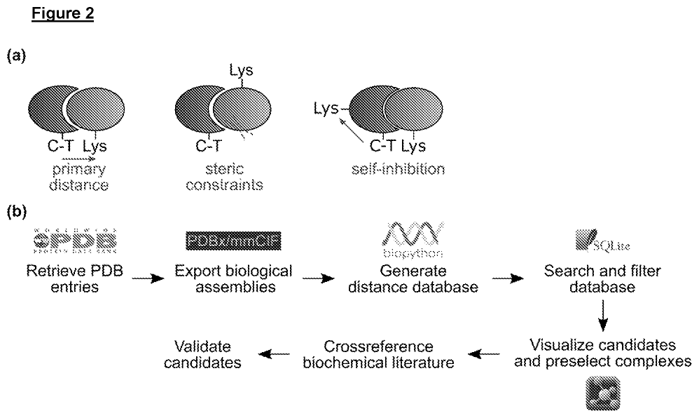

As noted above, in preferred embodiments, the polypeptides for conjugation are selected on the basis that they bind selectively and based on the distance from the C-terminal anhydride to the nearest nucleophile on the target polypeptide. Suitable polypeptide pairs may be selected using computer implemented methods as described in the Examples. For instance, tertiary and quaternary protein structures (e.g. from the Protein Data Bank (PDB)) may be screened to generate a database with distances from the most distal resolved residue (e.g. the residue at the C-terminus) in a given polypeptide to nucleophilic residues (e.g. lysine ε-amino groups) in the same structure (e.g. the same polypeptide or a different polypeptide in the quaternary structure). This database may be sorted and filtered, e.g. based on the distance between the most distal resolved residue and nucleophilic residues, and suitable polypeptide pairs may be verified by visualization and inspection in PyMOL (e.g. to evaluate the possibility of steric hindrance/accessibility and/or self-inhibition as shown in FIG. 2a) and selected for use in the claimed methods and uses. Representative examples of suitable polypeptide pairs obtained using the method described above are set out in Table 1 below.

TABLE 1

| PDB | | | Res. | | | 1° dist. |
|---|---|---|---|---|---|---|
| ID | Complex | Organism | (Å) | C-terminal atom | Target atom | (Å) |
| 1mox | Epidermal Growth Factor | Homo sapiens | 2.5 | Chain D (48 | Chain B (501 | 3.3 |
| | Receptor/Transforming Growth | | | res. long) ALA | res. long) LYS | |
| | Factor alpha | | | 50, atom C | 465, atom NZ | |
| 4zgy | Ornithine Decarboxylase/ | Homo sapiens | 2.6 | Chain B (125 | Chain A (383 | 3.5 |
| | Ornithine Decarboxylase | | | res. long) GLU | res. long) LYS | |
| | Antizyme | | | 219, atom C | 92, atom NZ | |
| 1ory | Flagellar protein FliS, Flagellin | Aquifex | 2.4 | Chain B (40 | Chain A (119 | 3.8 |
| | | aeolicus | | res. long) ARG | res. long) LYS | |
| | | | | 2518, atom C | 1028, atom NZ | |
| 2qac | Myosin A tail domain interacting | Plasmodium | 1.7 | Chain A (144 | Chain T (14 | 3.9 |
| | protein MTIP. Myosin-A | falciparum | | res. long) GLN | res. long) LYS | |
| | | | | 204, atom C | 813, atom NZ | |
| 1dml | DNA polymerase processivity | Human | 2.7 | Chain B (36 | Chain A (267 | 3.9 |
| | factor/DNA polymerase | herpesvirus 1 | | res. long) ALA | res. long) LYS | |
| | | | | 1235, atom C | 289, atom NZ | |
| 5yqz | Glucagon receptor, Endolysin, | Homo sapiens, | 3.0 | Chain P (28 | Chain R (558 | 4.1 |
| | Glucagon analogue | Enterobacteria | | res. long) THR | res. long) LYS | |
| | | phage T4 | | 29, atom C | 64, atom NZ | |
| 1syx | Spliceosomal U5 snRNP-specific | Homo sapiens | 2.3 | Chain B (62 | Chain A (135 | 4.3 |
| | 15 kDa protein/CD2 antigen | | | res. long) THR | res. long) LYS | |
| | cytoplasmic tail-binding protein 2 | | | 86, atom C | 125, atom NZ | |
| 1g0y | Interleukin-I receptor, Type I/ | Homo sapiens | 3.0 | Chain I (21 | Chain R (310 | 5.5 |
| | Antagonist peptide AF10847 | | | res. long) LEU | res. long) LYS | |
| | | | | 21, atom C | 95, atom NZ | |

C-terminal atom: selects carboxy C of last resolved residue in a given polypeptide chain, otherwise Cα, N or none. Target atom: on a chain other than the selected C-terminus, selects Nε (NZ) for lysine or αN for amino-terminus if resolved, otherwise Cα, N or none. 1° distance: distance between a C-terminus and an intermolecular target atom, i.e. the distance between lysine Nε (NZ) or amino-terminal N to C-terminal carboxy C on a different chain. Shown are the lowest 1° distances for each structure, with the corresponding C-terminal atoms and target atoms.

The equivalent process may be applied to any polypeptide of interest or portions thereof to identify suitable cognate polypeptides or portions thereof for use in the methods and uses of the invention, e.g. for use in domain (i) of the chimeric protein of the invention.

The process described above usually relies on the most distal resolved residue in a protein structure and its distance to a suitable nucleophilic group in the same structure. As not all amino acids in the protein structure may be fully resolved, the most distal resolved residue may not be at the C-terminus. Accordingly, when selecting polypeptides for use in the invention, it may be advantageous to use a portion of one or both polypeptides of a cognate pair. For instance, it may be useful to use only a portion of an endogenous polypeptide of a cognate pair in domain (i) of the chimeric protein based on the distance between the C-terminal amino acid of the portion and the nucleophilic group in the target polypeptide. In preferred embodiments, the portion of the endogenous polypeptide used in domain (i) of the chimeric protein is a functional polypeptide (e.g. retains at least some of the function of the full-length endogenous protein and is capable of binding non-covalently with the target polypeptide).

As discussed above, the polypeptide comprising the anhydride group may be used to direct the formation of a covalent bond, such as an amide bond or ester bond. In some embodiments, the amide bond is a peptide bond or an isopeptide bond.

A peptide bond is the amide bond which is formed when the carboxyl group of one amino acid becomes linked to the amino group of another. Thus, for instance, when the anhydride reacts with an N-terminal amine group (α-amine), a peptide bond may be formed.

The term “isopeptide bond” as used herein, refers to an amide bond between a carboxyl or carboxamide group and an amino group at least one of which is in an amino acid side chain. An isopeptide bond may form within a single protein or may occur between two polypeptides. Thus, an isopeptide bond may form intramolecularly within a single polypeptide or intermolecularly, i.e. between two peptide/polypeptide molecules. Typically, an isopeptide bond may occur between a lysine residue and an asparagine, aspartic acid, glutamine, or glutamic acid residue or the terminal carboxyl group of the polypeptide chain or may occur between the alpha-amino terminus of the polypeptide chain and an asparagine, aspartic acid, glutamine or glutamic acid. As discussed above, in the present invention, an anhydride group is formed on the aspartic acid or glutamic acid residue following proteolytic cleavage of the Asp-Pro or Glu-Pro bond which is directed to react with an amine group, e.g. by a proximity dependent interaction. In preferred embodiments of the invention, an isopeptide bond forms between a lysine residue (i.e. the ε-amine on a lysine residue) and an aspartate residue or between an α-amine group and an aspartate residue.

Typically, in order for covalent bond (e.g. an amide bond, such as an isopeptide bond) to form, the reactive residues, e.g. the reactive lysine and aspartate residues, should be positioned in close proximity to one another in space. However, the inventors have determined that the distance between the reactive residues may be larger than might be expected, e.g. based on the proximity of reactive residues in isopeptide proteins, i.e. proteins in which intramolecular isopeptide bonds form spontaneously (e.g. Spy0128 or FbaB of Streptococcus pyogenes). In isopeptide proteins the reactive residues typically are within about 4 Angstrom of each other in the folded protein (based on the distance between the C-epsilon atom in lysine and the C-gamma atom in aspartate).

Thus, when selecting polypeptides for use in the present invention, e.g. cognate pairs of polypeptides, it may be sufficient for the distance between the reactive residues, i.e. the C-terminal residue in the polypeptide in domain (i) of the chimeric protein (e.g. the binding polypeptide) and the functional group (e.g. Nε of lysine or αN of the amino-terminus) of the target polypeptide, to be within about 20 Angstrom (Å), e.g. within about 19, 18, 17, 16 or 15 Å, such as within about 1.0-20, 1.5-19, 2.0-18, 2.5-17, 3.0-16 or 3.5-15 Å.

As noted above, in a preferred embodiment, the polypeptides used in the methods, uses and chimeric protein of the invention are endogenous proteins or portions thereof based on the standard genetic code. Thus, the polypeptides may be produced recombinantly. In particular, the chimeric protein is a recombinantly produced protein. However, it will be evident that the target protein does not need to be produced recombinantly, although this is contemplated as an embodiment of the invention.

The nucleic acid molecules encoding the polypeptides used in the methods, uses and chimeric protein of the invention may be derived or obtained from any suitable source, e.g. any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa etc. In some embodiments, both of the polypeptides to be conjugated are synthetic polypeptides, e.g. produced recombinantly.

In some embodiments, the target molecule (e.g. target polypeptide) polypeptide for use in the invention may be derived or obtained from any suitable source. For instance, the polypeptide may be in vitro translated or purified from biological and clinical samples, e.g. any cell or tissue sample of an organism (eukaryotic, prokaryotic), or any body fluid or preparation derived therefrom, as well as samples such as cell cultures, cell preparations, cell lysates etc. Proteins may be derived or obtained, e.g. purified from environmental samples, e.g. soil and water samples or food samples are also included. The samples may be freshly prepared or they may be prior-treated in any convenient way e.g. for storage.

In some embodiments, the target polypeptide may be unpurified, or partially purified or isolated. For instance, the target polypeptide may be present in biological, clinical or environmental samples as described above. Alternatively viewed, biological, clinical or environmental samples as described above containing the target polypeptide may be used in the methods and uses of the invention. Thus, in some embodiments, the target polypeptide may be in its native or natural setting, e.g. on the surface of a cell or virus. Thus, for instance, the target polypeptide may be a transmembrane polypeptide (e.g. a receptor), membrane-bound polypeptide or viral coat protein.

The cell may be a prokaryotic or eukaryotic cell. In some embodiments, the cell is a eukaryotic (e.g. human) cell, such as a blood cell, e.g. red blood cell.

In some embodiments, the target polypeptide may be a modified polypeptide, e.g. linked to another molecule or structure. For instance, the target molecule may be provided as part of a nanoparticle, nanotube, polymer, virus-like particle, exosome, solid support or any combination thereof. In some embodiments, the target polypeptide may be conjugated to, or labelled with, a nucleic acid molecule, protein (e.g. antibody), peptide, small-molecule organic compound, fluorophore, metal-ligand complex or polysaccharide.

As a representative example, the polypeptides used in the methods, uses and chimeric protein of the invention may be enzymes, structural proteins, antibodies, antigens, prions, receptors, ligands, lectins, cytokines, chemokines, hormones and so on or any combination thereof. In some preferred embodiments, the polypeptides are cognate pairs of polypeptides, e.g. antibody (or antigen-binding portion thereof, e.g. scFv) and antigen/hapten, ligand and receptor, components of a protein (e.g. enzymatic) complex, lectin and glycosylated polypeptide etc.

In some embodiments, the polypeptide in domain (i) of the chimeric protein is a growth factor, cytokine or chemokine or a functional portion or derivative thereof. For instance, the polypeptide may be selected from any one of TGFα, epigen, epiregulin, EGF, HB-EGF, TGFβ, TNFα, IL1RA, IL-β, IL-2, IL-4, IL-5, IL-6, IL-7, IL-8 (CXCL8), IL-9, IL-10, IL-12, IL-13, IL-15, IL-17, CCL11, BasicFGF, G-CSF, GM-CSF, INFα, INFγ, CXCL10, CCL2, CCL3, CCL4, PDGF-β, CCLS, VEGF or a functional portion or derivative thereof. In some preferred embodiments, the growth factor is TGFα. Thus, in some embodiments, the chimeric protein comprises N-terminus to C-terminus:

(i) a domain comprising a polypeptide having an amino acid sequence as set forth in SEQ ID NO: 17; and

(ii) a domain comprising a self-processing module comprising:

(1) an amino acid sequence as set forth in any one of SEQ ID NOs: 1-4;

(2) a portion of (1) comprising an amino acid sequence as set forth in any one of SEQ ID NOs: 5-8;

(3) an amino acid sequence with at least 60% sequence identity to an amino acid sequence as set forth in any one of SEQ ID NOs: 1-4; or

(4) a portion of (3) comprising an amino acid sequence with at least 60% sequence identity to an amino acid sequence as set forth in any one of SEQ ID NOs: 5-8,

wherein the first (N-terminal) amino acid of the domain comprising a self-processing module is an aspartate or glutamate and the second amino acid of the domain comprising a self-processing module is proline;

and wherein the self-processing module cleaves the peptide bond between the first and second amino acids of the domain comprising a self-processing module under suitable conditions.

In some embodiments, the SPM may be selected from any of the variants and portions defined above.

In some embodiments, the chimeric protein comprises an amino acid sequence as set forth in SEQ ID NO: 16.

Thus, in some embodiments, the target polypeptide is a cytokine or chemokine receptor or a binding portion thereof. For instance, in some embodiments, the target polypeptide is epidermal growth factor receptor (EGFR).

It will be evident that the polypeptide in domain (i) of the chimeric protein (e.g. binding polypeptide) is not from the protein from which the self-processing module is derived.

In some embodiments, methods and uses of the invention may be used to create a homodimer, i.e. the same polypeptide or portions thereof may be linked together.

The term “endogenous polypeptide” refers to a native or natural polypeptide originating from an organism, tissue, or cell. Thus, the amino acid sequence of a polypeptide that is identical to a polypeptide or portion thereof from an organism, tissue or cell may be viewed as an endogenous polypeptide, even if the portion of the polypeptide does not occur naturally. As noted above, the polypeptide in domain (i) of the chimeric protein preferably comprises an amino acid sequence of an endogenous polypeptide. However, upon cleavage of the chimeric protein by the self-processing module, the resulting polypeptide contains an aspartate or glutamate that is not present at the equivalent position in the amino acid sequence of the corresponding endogenous polypeptide or portion thereof. The resulting polypeptide will also contain a peptide linker as defined above, if present in the chimeric protein.

An “equivalent position” in the polypeptide or product the invention is determined by reference to the amino acid sequence of the corresponding endogenous polypeptide. The equivalent (homologous or corresponding) position can be readily deduced by lining up the sequence of the polypeptide or product of the invention and the sequence of the endogenous polypeptide or portion thereof, for example using a BLAST algorithm, e.g. using the BLASTP algorithm.

As described above, in some embodiments, the polypeptide comprising an anhydride group may react with a functional group in a non-polypeptide molecule, e.g. a sugar or lipid. In some embodiments, the sugar or lipid may be linked to a polypeptide by a covalent bond (directly or indirectly). Thus, where the polypeptide comprising an anhydride group reacts with a functional group in a non-polypeptide molecule linked to a polypeptide by a covalent bond, an equivalent position refers to the amino acid to which the non-polypeptide molecule is linked. Similarly, an equivalent position in a carbohydrate (e.g. oligosaccharide) or lipid molecule may be determined by reference to the structure of the units (e.g. sugars or carbons) in the endogenous molecules.

In some embodiments, at least one of the polypeptides to be conjugated (e.g. the binding polypeptide/first polypeptide) has a therapeutic or prophylactic effect or utility, e.g. a cytokine, toxin, antigen. Thus, the chimeric protein and products of the invention may find utility in therapy and diagnostics.

As a representative example, the polypeptide in domain (i) of the chimeric protein may be a cytokine with utility in tumour therapy, e.g. capable of inhibiting the growth of a tumour and/or to target the tumour cells for destruction by the immune system. In this respect, the systemic administration of cytokines for the treatment of tumours is problematic because the effects are not limited to the tumour, often resulting in side-effects/toxicity. Even local (e.g. intratumoral) administration is problematic as the cytokine would normally diffuse elsewhere in the body, again leading to toxic effects, or be cleared from the tumour, e.g. by uptake of the target cells and intracellular proteolysis.

Thus, a chimeric protein of the invention may comprise a therapeutic polypeptide (e.g. cytokine) in domain (i) (e.g. a cytokine with direct or indirect anti-tumour activity), which is capable of binding specifically to the tumour, e.g. to a tumour-specific antigen and/or in the extracellular matrix of the tumour (i.e. the target polypeptide). In some embodiments, domain (i) may comprise a binding domain that mediates the interaction between the therapeutic polypeptide (e.g. cytokine) and the tumour-specific antigen. Administration of the chimeric protein, e.g. systemically or intratumorally, allows the chimeric protein to bind to the target polypeptide under conditions that induce the autoproteolytic cleavage of the chimeric protein and subsequent conjugation of the therapeutic polypeptide to the target polypeptide. As noted above, extracellular concentrations of Ca²⁺ are sufficient to activate the SPM. Conjugation of the therapeutic polypeptide to the extracellular matrix of the tumour would result in the therapeutic polypeptide being trapped inside the tumour and resistant to endocytosis, enabling the therapeutic polypeptide (e.g. cytokine) to remain functional without, or with minimal, toxic effects. It will be evident that a therapeutic polypeptide comprising a reactive anhydride obtained from the chimeric protein could be used directly, e.g. when administered directly to the disease site, i.e. intratumorally. In this respect, it may be necessary to generate the polypeptide comprising the anhydride group locally (i.e. in proximity to the patient) such that it can be administered immediately as defined above, e.g. within 20 minutes of the formation of the anhydride group, e.g. within 15, 10, 9, 8, 7, 6 or 5 minutes of the formation of the anhydride group.

In another representative embodiment, the chimeric protein may be used to conjugate an immunosuppressive polypeptide (e.g. cytokine) to an organ for transplantation to reduce the risk of rejection, e.g. graft versus host disease. For instance, the immunosuppressive polypeptide (e.g. cytokine) may reduce or discourage the infiltration of lymphocytes into the transplanted organ and/or to modulate the phenotype of lymphocytes infiltrating the transplanted organ.

Similarly, the chimeric protein (or reactive polypeptides obtained therefrom) may be used to conjugate therapeutic polypeptides to red blood cells. The isopeptide bond generated by the method of the invention is irreversible, which is a significant advantage over existing non-covalent approaches (e.g. antibody anchoring) of coupling molecules to red blood cells. Thus, the present invention would enable the therapeutic polypeptide to be effective for a longer period of time, e.g. the life of the red blood cells.

In a further representative embodiment, the chimeric protein (or reactive polypeptides obtained therefrom) may be used to conjugate polypeptides to exosomes, e.g. for drug delivery. For example, the polypeptides may be used to target exosomes comprising a therapeutically active agent to target cells, e.g. diseased cells.

In another embodiment, the chimeric protein (or reactive polypeptides obtained therefrom) may be used to anchor polypeptides (e.g. antigens) to virus-like particles for vaccine assembly.

Another utility of the chimeric protein (or reactive polypeptides obtained therefrom) may be in the mechanical cross-linking of the extracellular matrix to promote joint, tendon or ligament repair. Similarly, anchoring signalling polypeptides to the extracellular matrix may find utility in wound repair.

In yet another embodiment, the chimeric protein (or reactive polypeptides obtained therefrom) may be used to conjugate signalling polypeptides to surface receptors for activation or inhibition of the receptors. Covalent conjugation may result in an extended pharmacokinetic profile.

Thus, in another aspect, the invention provides a pharmaceutical composition comprising: (a)(i) a chimeric protein as defined herein; (ii) a polypeptide comprising an anhydride group as defined herein or composition containing said polypeptide as defined above; or (iii) a product as defined herein, and (b) one or more pharmaceutically acceptable excipients and/or diluents.

Thus, in a further aspect, the invention provides a (i) chimeric protein as defined herein; (ii) polypeptide comprising an anhydride group as defined herein or composition containing said polypeptide as defined above; (iii) product as defined herein; or (iv) pharmaceutical composition as defined herein, for use in therapy or diagnosis.

Alternatively viewed, the invention provides a method of treating a disease in a subject comprising administering to a subject in need thereof a therapeutically effective amount of a (i) chimeric protein as defined herein; (ii) polypeptide comprising an anhydride group as defined herein or composition containing said polypeptide as defined above; (iii) product as defined herein; or (iv) pharmaceutical composition as defined herein, thereby treating the disease.

As noted above, in some embodiments, the polypeptide comprising an anhydride group is produced locally (i.e. in the vicinity of the subject) and administered to the subject immediately. Thus, in some embodiments, the method further comprises a step of producing the polypeptide comprising an anhydride group, e.g. using the methods described above.

“Pharmaceutically acceptable” refers to ingredients that are compatible with other ingredients used in the methods or uses of the invention as well as being physiologically acceptable to the recipient.

As defined herein “treating” or “treatment” as used herein refers broadly to any effect or step (or intervention) beneficial in the management of a clinical condition or disorder. Treatment therefore may refer to reducing, alleviating, ameliorating, slowing the development of, or eliminating one or more symptoms of the disease which is being treated, relative to the symptoms prior to treatment, or in any way improving the clinical status of the subject. A treatment may include any clinical step or intervention which contributes to, or is a part of, a treatment programme or regimen.

A treatment may include delaying, limiting, reducing or preventing the onset of one or more symptoms of the disease, for example relative to the disease or symptom prior to the treatment. Thus, treatment explicitly includes both absolute prevention of occurrence or development of a symptom of the disease, and any delay in the development of the disease or symptom, or reduction or limitation on the development or progression of the disease or symptom.

The “subject” or “patient” is an animal (i.e. any human or non-human animal), preferably a mammal, most preferably a human.

The therapeutic agents described herein (e.g. the chimeric protein) may be administered to the subject using any suitable means and the route of administration will depend on the therapeutic agent and disease to be treated. In some embodiments, the therapeutic agent is administered systemically. In some embodiments, the therapeutic agent is administered locally.

“Systemic administration” includes any form of non-local administration in which the agent is administered to the body at a site other than the disease site, directly adjacent to, or in the local vicinity of, the disease site, resulting in the whole body receiving the administered agent. Conveniently, systemic administration may be via enteral delivery (e.g. oral) or parenteral delivery (e.g. intravenous, intramuscular or subcutaneous).

“Local administration” refers to administration of the agent to the body at the site of the disease, at a site directly adjacent to the site of the disease, or in the local vicinity of the disease site, resulting in only part of the body receiving the administered agent. Local administration may be via parenteral delivery (e.g. intratumoral injection, intra-articular injection).

The excipient may include any excipients known in the art, for example any carrier or diluent or any other ingredient or agent such as buffer, antioxidant, chelator, binder, coating, disintegrant, filler, flavour, colour, glidant, lubricant, preservative, sorbent and/or sweetener etc.

The pharmaceutical compositions described herein may be provided in any form known in the art, for example as a liquid, suspension, solution, dispersion, emulsion or any mixtures thereof.

While therapeutic and diagnostic methods and uses are contemplated herein, the chimeric protein and associated products of the invention also find utility in numerous in vitro methods and uses. For instance, the method may involve conjugation of polypeptides in vitro, such as conjugation of a polypeptide to a cell (e.g. red blood cell) in vitro. In some embodiments, the conjugation products obtained from in vitro methods and uses may find utility in the therapeutic methods and uses as defined above. Thus, in some embodiments, the methods and uses described herein may be viewed as ex vivo methods and uses.

Representative examples of in vitro utilities of the invention include the production of biomaterials or in anchoring polypeptides to materials, e.g. nanopores for nucleic acid sequencing. For instance, a polypeptide comprising an anhydride group obtained from the chimeric protein may be linked to a surface comprising an amine group, e.g. by contacting the polypeptide comprising an anhydride group with the surface comprising amine, hydroxylamine or hydrazide groups under conditions suitable to form a covalent bond. Thus, in some embodiments, the target molecule may be an amine (e.g. a molecule comprising an amine group) linked to a surface (e.g. solid phase/support). In some embodiments, the amine group on the surface is part of peptide or polypeptide immobilised on the surface.

The term “target polypeptide” may be replaced herein with the term “target molecule” in some embodiments, e.g. where the chimeric protein is used to mediate the conjugation of a polypeptide to a non-polypeptide entity, such as a solid support, lipid or carbohydrate (e.g. sugar, oligosaccharide).

In some embodiments, it may be useful to immobilise the chimeric protein of the invention on a solid substrate (i.e. a solid phase or solid support), e.g. to generate a polypeptide comprising a reactive anhydride group on a solid support, and this may be achieved in any convenient way. Thus, the manner or means of immobilisation and the solid support may be selected, according to choice, from any number of immobilisation means and solid supports as are widely known in the art and described in the literature. Thus, the chimeric protein may be directly bound to the support, for example via a domain or moiety of the protein (e.g. chemically cross-linked). In some embodiments, the chimeric protein may be bound indirectly by means of a linker group, or by an intermediary binding group(s) (e.g. by means of a biotin-streptavidin interaction). Thus, the chimeric protein may be covalently or non-covalently linked to the solid support. The linkage may be a reversible (e.g. cleavable) or irreversible linkage. Thus, in some embodiments, the linkage may be cleaved enzymatically, chemically, or with light, e.g. the linkage may be a light-sensitive linkage.

Thus, in some embodiments, a chimeric protein may be provided with means for immobilisation (e.g. an affinity binding partner, e.g. biotin or a hapten) capable of binding to its binding partner, i.e. a cognate binding partner (e.g. streptavidin or an antibody) provided on the support. In some embodiments, the means for immobilisation may form a further domain of the chimeric protein or may be viewed as being part of one of the domains described above, e.g. part of the domain containing the SPM. In some embodiments, the interaction between the chimeric protein and the solid support must be robust enough to allow for washing steps, i.e. the interaction between the chimeric protein and solid support is not disrupted (significantly disrupted) by the washing steps. For instance, it is preferred that with each washing step, less than 5%, preferably less than 4, 3, 2, 1, 0.5 or 0.1% of the chimeric protein is removed or eluted from the solid phase.

In some embodiments, the chimeric protein of the invention may comprise additional sequences (e.g. peptide/polypeptide tags to facilitate purification of the polypeptide prior to use in the process and for use of the invention discussed herein). Any suitable purification moiety or tag may be incorporated into the polypeptide and such moieties are well known in the art. For instance, in some embodiments, the polypeptide may comprise a peptide purification tag or moiety, e.g. a His-tag, C-tag, SpyTag sequence. Such purification moieties or tags may be incorporated at any position within the chimeric protein. In some preferred embodiments, a purification moiety is located at or towards (i.e. within 5, 10, 15, 20 amino acids of) the N- or C-terminus of the protein. In some embodiments, a purification tag is incorporated in domain (i) of the chimeric protein, e.g. to facilitate purification of the conjugation product. In some embodiments, a purification tag is incorporated in domain (ii) of the chimeric protein (the domain comprising the SPM), e.g. to facilitate removal of the cleaved self-processing module.

In some embodiments, the chimeric protein may be used to isolate (e.g. purify) a recombinant polypeptide, e.g. using affinity chromatography. For instance, the polypeptide desired for isolation (e.g. purification) forms domain (i) of the chimeric protein. A sample comprising the chimeric protein (e.g. the lysate of cells in which the chimeric protein was produced) may be contacted with a solid support comprising means to selectively bind the chimeric protein under conditions that enable the chimeric protein to selectively bind to said solid support, thereby forming a non-covalent complex between the chimeric protein and the solid support. As noted above, the chimeric protein may comprise an affinity tag that binds to its binding partner immobilised (directly or indirectly) on the solid support. The solid support may be washed with a buffer (e.g. as defined below) to remove unbound molecules followed by activation of the SPM (e.g. by the addition of buffer containing calcium ions as described above) to promote cleavage of the chimeric protein, thereby releasing the desired polypeptide (e.g. in an isolated form, i.e. isolated (e.g. purified) from other components in the sample).

In embodiments where the chimeric protein is immobilised on the solid support via an interaction with domain (i) of the chimeric protein, the desired polypeptide will be retained on the solid support following cleavage of the chimeric protein. The solid support may be subjected to further wash steps prior to dissociation (e.g. elution) of the desired polypeptide from the solid support.

In embodiments where the chimeric protein is immobilised on the solid support via an interaction with the domain of the chimeric protein containing the SPM (e.g. via an affinity tag), the desired polypeptide will be released from the solid support following cleavage of the chimeric protein. The solid support may be subjected to further wash steps to maximise the release and yield of the desired polypeptide. In embodiments where the desired polypeptide is released and/or collected in more than one fraction, it may be advantageous to pool and/or concentrate the fractions to obtain the isolated (e.g. purified) polypeptide.

In some embodiments, it may be advantageous to subject the isolated (e.g. purified) polypeptide to conditions sufficient to allow hydrolysis of the anhydride group. This may be achieved on the solid support or following dissociation (e.g. elution) from the solid support. In this respect, the isolated (e.g. purified) polypeptide will contain a C-terminal aspartate or glutamate residue.

Thus, in some embodiments, the invention provides the use of chimeric protein to isolate (e.g. purify) a desired polypeptide, wherein the chimeric protein comprises N-terminus to C-terminus:

(i) a domain comprising the desired polypeptide; and

(ii) a domain comprising a self-processing module comprising:

(1) an amino acid sequence as set forth in SEQ ID NO: 1;

(2) a portion of (1) comprising an amino acid sequence as set forth in SEQ ID NO: 5;

(3) an amino acid sequence with at least 80% sequence identity to an amino acid sequence as set forth in SEQ ID NO: 1 or 2; or

(4) a portion of (3) comprising an amino acid sequence with at least 80% sequence identity to an amino acid sequence as set forth in SEQ ID NO: 5 or 6,

wherein the amino acid sequence comprises aspartate or glutamate at position 1, proline at position 2 and one or more of the following:
1) alanine at position 17;
2) alanine at position 23;
3) arginine at position 28;
4) glutamine at position 30;

and wherein the self-processing module cleaves the peptide bond between the first and second amino acids of the domain comprising a self-processing module under suitable conditions.

Alternatively viewed, the invention provides a method of isolating (e.g. purifying) a desired polypeptide comprising:

a) providing a sample comprising a chimeric protein, wherein the chimeric protein comprises N-terminus to C-terminus:

(i) a domain comprising the desired polypeptide; and

(ii) a domain comprising a self-processing module comprising:

(1) an amino acid sequence as set forth in SEQ ID NO: 1;

(2) a portion of (1) comprising an amino acid sequence as set forth in SEQ ID NO: 5;

(3) an amino acid sequence with at least 80% sequence identity to an amino acid sequence as set forth in SEQ ID NO: 1 or 2; or

(4) a portion of (3) comprising an amino acid sequence with at least 80% sequence identity to an amino acid sequence as set forth in SEQ ID NO: 5 or 6,

wherein the amino acid sequence comprises aspartate or glutamate at position 1, proline at position 2 and one or more of the following:
1) alanine at position 17;
2) alanine at position 23;
3) arginine at position 28;
4) glutamine at position 30;

and wherein the self-processing module cleaves the peptide bond between the first and second amino acids of the domain comprising a self-processing module under suitable conditions;

b) contacting the sample of a) with a solid support under conditions that enable said chimeric protein to selectively bind to said solid support, thereby forming a non-covalent complex between said chimeric protein and the solid support;

c) washing the solid support with a buffer;

d) inducing the self-processing module to cleave the peptide bond between the proline residue and the aspartate or glutamate residue (i.e. between residues 1 and 2) to release the desired polypeptide;

e) separating the desired polypeptide from the solid substrate.

In some embodiments, the chimeric polypeptide binds to the solid support via an interaction between an affinity tag in the polypeptide and its cognate binding partner immobilised on the solid support. In some embodiments, the affinity tag is a peptide tag in the domain of the chimeric protein containing the SPM. In some embodiments, the peptide tag is located at the C-terminus of the chimeric protein and/or SPM.

In some embodiments, separating the desired polypeptide from the solid support may comprise separating the solution containing the desired polypeptide from the solid support.

In some embodiments, separating the desired polypeptide from the solid support may comprise a step of disrupting the non-covalent interaction between the desired polypeptide and the solid support (i.e. dissociating (e.g. eluting) the desired polypeptide from the solid support) prior to the step of separating the solution containing the desired polypeptide from the solid support. In these embodiments, it may be advantageous to include a wash step after step (d) (i.e. inducing (activating) the SPM) to remove the cleaved SPM. The wash steps may use any suitable conditions, i.e. conditions that do not substantially disrupt the non-covalent interaction between the desired polypeptide and the solid support, e.g. such that less than 5%, preferably less than 4, 3, 2, 1, 0.5 or 0.1% of the desired polypeptide is removed or eluted from the solid phase.

Similarly, step (c) may use any suitable conditions, i.e. conditions that do not substantially disrupt the non-covalent interaction between the chimeric protein and the solid support, e.g. such that less than 5%, preferably less than 4, 3, 2, 1, 0.5 or 0.1% of the chimeric protein is removed or eluted from the solid phase.

In some embodiments, the method comprises a step of pooling and/or concentrating the solution containing the desired polypeptide (i.e. the solution obtained from step (e)).

In some embodiments, the solution containing the desired polypeptide (i.e. the solution obtained from step (e)) may be subjected to further purification steps.

The sample used in the method and use described above (i.e. comprising the chimeric protein containing a desired polypeptide) may be from any biological or clinical sample, e.g. any cell or tissue sample of an organism (eukaryotic, prokaryotic), or any body fluid or preparation derived therefrom, as well as samples such as cell cultures, cell preparations, cell lysates etc. The samples may be freshly prepared or they may be prior-treated in any convenient way e.g. for storage.

The solid support (phase or substrate) may be any of the well-known supports or matrices which are currently widely used or proposed for immobilisation, separation etc. These may take the form of particles (e.g. beads which may be magnetic, para-magnetic or non-magnetic), sheets, gels, filters, membranes, fibres, capillaries, slides, arrays or microtitre strips, tubes, plates or wells etc. In some embodiments, the solid support comprises nanopores.

The support may be made of glass, silica, metal, latex or a polymeric material. Suitable are materials presenting a high surface area for binding of the chimeric protein. Such supports may have an irregular surface and may be for example porous or particulate, e.g. particles, fibres, webs, sinters or sieves. Particulate materials, e.g. beads are useful due to their greater binding capacity, particularly polymeric beads.

Conveniently, a particulate solid support used according to the invention may comprise spherical beads. The size of the beads is not critical, but they may for example be of the order of diameter of at least 1 and preferably at least 2 μm, and have a maximum diameter of preferably not more than 10, and e.g. not more than 6 μm.

Monodisperse particles, that is those which are substantially uniform in size (e.g. size having a diameter standard deviation of less than 5%) have the advantage that they provide very uniform reproducibility of reaction.

However, to aid manipulation and separation, magnetic beads are advantageous. The term “magnetic” as used herein means that the support is capable of having a magnetic moment imparted to it when placed in a magnetic field, and thus is displaceable under the action of that field. In other words, a support comprising magnetic particles may readily be removed by magnetic aggregation, which provides a quick, simple and efficient way of separating the particles following the isopeptide bond formation steps.

It will be evident that immobilising the chimeric protein on a solid support may facilitate the methods and uses described herein, e.g. in conjugating polypeptides. For instance, immobilising the chimeric protein on a solid support allows the protein to be incubated with a target protein under conditions suitable for non-covalent interaction of the chimeric protein with the target protein as described above. Excess target polypeptide and other unbound (e.g. non-cognate molecules) may be removed by washing the solid support under suitable conditions, followed by activation of the SPM to promote the formation of the isopeptide bond between the first and second polypeptides. Thus, in some embodiments, the method is performed using a heterogeneous format (i.e. using a solid phase).

Notably however, a wash step is optional, as the specific non-covalent interaction between the first and second polypeptides (binding and target polypeptides) may be sufficient to direct the proximity based reaction with sufficient specificity without the need for a washing step. Thus, in some embodiments, the method is performed using a homogeneous format (i.e. in solution).

Thus, in some embodiments, the method of conjugating a first polypeptide to a second polypeptide via an isopeptide bond comprises:

(a) providing a chimeric protein comprising:

(i) a domain comprising the first polypeptide; and

(ii) a domain comprising a self-processing module that contains an N-terminal dipeptide of aspartate or glutamate and proline (D/E-P),

wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions;

(b) immobilising the chimeric protein on a solid support (e.g. via immobilisation moiety in domain (i) or (ii), such as a peptide tag as described above);

(c) contacting the solid support comprising the chimeric protein of (b) with the second polypeptide, wherein the second polypeptide binds non-covalently to (i);

(d) washing the solid support under conditions suitable to disrupt non-specific interactions; and

(e) inducing the self-processing module to cleave the peptide bond between the proline residue and the aspartate or glutamate residue to release the first polypeptide and generate an anhydride group on the aspartate or glutamate residue that reacts with an amine group on the second polypeptide to form an isopeptide bond, thereby conjugating the first and second polypeptides.

In some embodiments, step (a) may comprise providing an immobilised chimeric protein, thereby obviating the need for step (b).

The step of washing the solid support may utilise any suitable buffer and this will depend on the properties of the polypeptides to be conjugated. Furthermore, the step of washing the solid support may be repeated multiple times, e.g. 2, 3, 4, 5 or more times. Alternatively viewed, in some embodiments the method comprises multiple wash steps, wherein the same or different washing conditions may be used in each step.

Where the solid support comprises beads (e.g. agarose-based beads) the volume of buffer used in the wash steps may be at least about 2 times the volume of the beads, e.g. at least about 3, 4, 5, 6, 7, 8, 9 or 10 times the volume of the beads.

The temperature of the washing steps may be determined readily by a person of skill in the art based on routine experimentation and may depend on the nature of the polypeptides being conjugated. In some embodiments, the washing steps are performed at 10° C. or less, e.g. 9, 8, 7, 6, 5 or 4° C. or less.

Whilst it may be useful to immobilise the chimeric protein of the invention on a solid support prior to contact with the sample comprising the target molecule, it will be evident that this is not essential. For instance, the binding of the chimeric protein and the target molecule may take place in solution, which is subsequently applied to a solid support or solid phase, e.g. column, for subsequent washing and conjugation steps. In some embodiments, the chimeric protein:target molecule complex may be applied to the solid phase under conditions suitable to immobilise the complex on the solid phase via the chimeric protein or the target molecule (e.g. an immobilisation domain in or on the chimeric protein or the target molecule), washed under suitable conditions and subsequently subjected to one or more of the conditions mentioned above to induce the SPM and promote the formation of the isopeptide bond.

As noted above, an advantage of the present invention arises from the fact that the chimeric protein and target polypeptide may be completely genetically encoded. Thus, in a further aspect, the invention provides a nucleic acid molecule encoding a chimeric protein as defined above.

The nucleic acid molecules of the invention may be made up of ribonucleotides and/or deoxyribonucleotides as well as synthetic residues, e.g. synthetic nucleotides, that are capable of participating in Watson-Crick type or analogous base pair interactions. Preferably, the nucleic acid molecule is DNA or RNA.

The nucleic acid molecules described above may be operatively linked to an expression control sequence, or a recombinant DNA cloning vehicle or vector containing such a recombinant DNA molecule. This allows cellular expression of the chimeric protein of the invention as a gene product, the expression of which is directed by the gene(s) introduced into cells of interest. Gene expression is directed from a promoter active in the cells of interest and may be inserted in any form of linear or circular nucleic acid (e.g. DNA) vector for incorporation in the genome or for independent replication or transient transfection/expression. Suitable transformation or transfection techniques are well described in the literature. Alternatively, the naked nucleic acid (e.g. DNA or RNA, which may include one or more synthetic residues, e.g. base analogues) molecule may be introduced directly into the cell for the production of polypeptides of the invention. Alternatively the nucleic acid may be converted to mRNA by in vitro transcription and the relevant proteins may be generated by in vitro translation.

Appropriate expression vectors include appropriate control sequences such as for example translational (e.g. start and stop codons, ribosomal binding sites) and transcriptional control elements (e.g. promoter-operator regions, termination stop sequences) linked in matching reading frame with the nucleic acid molecules of the invention. Appropriate vectors may include plasmids and viruses (including both bacteriophage and eukaryotic viruses). Suitable viral vectors include baculovirus and also adenovirus, adeno-associated virus, herpes and vaccinia/pox viruses. Many other viral vectors are described in the art. Examples of suitable vectors include bacterial and mammalian expression vectors.

As noted above, the chimeric protein of the invention may comprise additional sequences (e.g. peptide/polypeptide tags to facilitate immobilisation of the chimeric protein or purification of the products of the method, i.e. the conjugated binding and target polypeptides or the desired polypeptide) and thus the nucleic acid molecule may conveniently be fused with DNA encoding an additional peptide or polypeptide, e.g. His-tag, C-tag, SpyTag, to produce the chimeric protein on expression.

Thus viewed from a further aspect, the present invention provides a vector, preferably an expression vector, comprising a nucleic acid molecule as defined above.

Other aspects of the invention include methods for preparing recombinant nucleic acid molecules according to the invention, comprising inserting nucleic acid molecule of the invention encoding the chimeric protein (or the SPM) of the invention into vector nucleic acid.

Nucleic acid molecules of the invention, preferably contained in a vector, may be introduced into a cell by any appropriate means. Suitable transformation or transfection techniques are well described in the literature. Numerous techniques are known and may be used to introduce such vectors into prokaryotic or eukaryotic cells for expression. Preferred host cells for this purpose include insect cell lines, yeast, mammalian cell lines or E. coli. The invention also extends to transformed or transfected prokaryotic or eukaryotic host cells containing a nucleic acid molecule, particularly a vector as defined above.

In some embodiments, the chimeric protein produced in a host cell is located in the cytosol, where conditions are not suitable for activation of the SPM, e.g. the calcium concentration is not sufficient to induce cleavage of the D-P or E-P bond. However, in some embodiments, it may be advantageous to target the chimeric protein to an intracellular compartment with the required calcium concentration, e.g. endoplasmic reticulum, or outside the cell (e.g. target the chimeric protein to the secretory pathway). For instance, this may be particularly useful when the target polypeptide is co-expressed in the host cell and located in an intracellular compartment with the required calcium concentration, e.g. endoplasmic reticulum, or outside the cell. Thus, in some embodiments, the steps of contacting the chimeric polypeptide with the target polypeptide and activating the SPM may be intracellular or in vivo. Thus, in some embodiments, the chimeric protein may comprise a signal peptide that functions to translocate the protein to an intracellular compartment or into the extracellular matrix (i.e. targets the chimeric protein or the product of the invention for secretion), e.g. to a cellular location (e.g. an intracellular compartment) comprising the target polypeptide and the required calcium concentration, e.g. endoplasmic reticulum, or outside the cell.

However, in embodiments where it is desirable to isolate the chimeric protein intact (e.g. for reaction with the target polypeptide) it will be important to ensure that the chimeric protein is not targeted to a cellular location upon expression in the host cell that would activate the SPM. Thus, where the endogenous polypeptide selected for use in domain (i) of the chimeric protein contains a signal peptide (e.g. a signal peptide that would translocate the polypeptide to a compartment containing the required calcium concentration to activate the SPM, e.g. where the polypeptide is a secreted or transmembrane protein), it may be preferable to use only a portion of the endogenous polypeptide in the chimeric protein (i.e. a portion that does not contain the signal peptide). Alternatively, it may be preferable to express the chimeric protein in a prokaryotic cell.

Thus, in another aspect, there is provided a recombinant host cell containing a nucleic acid molecule and/or vector as described above. The host cell may be a prokaryotic or eukaryotic cell. In some embodiments, the host cell is a prokaryotic cell.

By “recombinant” is meant that the nucleic acid molecule and/or vector has been introduced into the host cell. The host cell may or may not naturally contain an endogenous copy of the nucleic acid molecule, but it is recombinant in that an exogenous or further endogenous copy of the nucleic acid molecule and/or vector has been introduced.

A further aspect of the invention provides a method of preparing a chimeric protein of the invention as hereinbefore defined, which comprises culturing a host cell containing a nucleic acid molecule as defined above, under conditions whereby said nucleic acid molecule encoding said chimeric protein is expressed and recovering said chimeric protein. The expressed chimeric protein forms a further aspect of the invention.

In some embodiments, the chimeric protein of the invention, or for use in the method and uses of the invention, may be generated synthetically, e.g. by ligation of amino acids or smaller synthetically generated peptides, or more conveniently by recombinant expression of a nucleic acid molecule encoding said chimeric protein as described hereinbefore.

Nucleic acid molecules of the invention may be generated synthetically by any suitable means known in the art.

Thus, the chimeric protein and/or target polypeptide of the invention may be an isolated, purified, recombinant or synthesised protein or polypeptide.

Similarly, the nucleic acid molecules of the invention may be an isolated, purified, recombinant or synthesised nucleic acid molecule.

Thus, alternatively viewed, the polypeptides and nucleic acid molecules of the invention are preferably non-native, i.e. non-naturally occurring, molecules.

Standard amino acid nomenclature is used herein. Thus, the full name of an amino acid residue may be used interchangeably with one letter code or three letter abbreviations. For instance, lysine may be substituted with K or Lys, isoleucine may be substituted with l or lle, and so on. Moreover, the terms aspartate and aspartic acid, and glutamate and glutamic acid are used interchangeably herein and may be replaced with Asp or D, or Glu or E, respectively.

In a further embodiment, the invention provides a kit, particularly a kit for use in the methods and uses of the invention, e.g. for conjugating two polypeptides via an isopeptide bond, wherein said kit comprises:

(a) a chimeric protein as defined above (e.g. a container comprising the chimeric protein); and optionally

(b) a target polypeptide as defined above, a solid support upon which the chimeric protein may be immobilised, and/or a buffer suitable to induce the autoproteolytic activity of the SPM.

The invention will now be described in more detail in the following non-limiting Examples with reference to the following drawings:

FIG. 1 shows: (a) a schematic of the FrpC self-processing module (SPM), which catalyzes autoproteolytic cleavage at an Asp-Pro bond, induced by calcium. The resultant anhydride enables protein-protein crosslinking via reaction with nucleophilic side-chains; (b) a schematic of the chimeric protein (NeissLock probe) and its utility to conjugate two polypeptides. The SPM is recombinantly fused to a binding protein which docks with the target protein. Adding calcium promotes generation of the anhydride and the binding protein then can form a covalent bond to the target protein; (c) a photograph of an SDS-PAGE gel with Coomassie staining showing a time-course of SPM cleavage with Ala preceding Asp-Pro; and (d) a histogram of SPM cleavage rate with each residue before Asp-Pro, moving from the least cleaved residue at 60 min on the left to the most cleaved residue on the right (mean of triplicate±1 s.d.; some error bars are too small to be visible).

FIG. 2 shows (a) a diagrammatic representation of the considerations for binder/target complex selection. The target protein should have a lysine or N-terminal amine in proximity and sterically accessible to the C-terminus of the binder protein, to enable reaction with the anhydride formed during activation. To avoid quenching by self-reaction, the binder protein should not feature a lysine close to its own C-terminus; and (b) a flow chart of the disCrawl distance database pipeline, i.e. the computer implemented method of selecting polypeptides for use in the method of the invention.

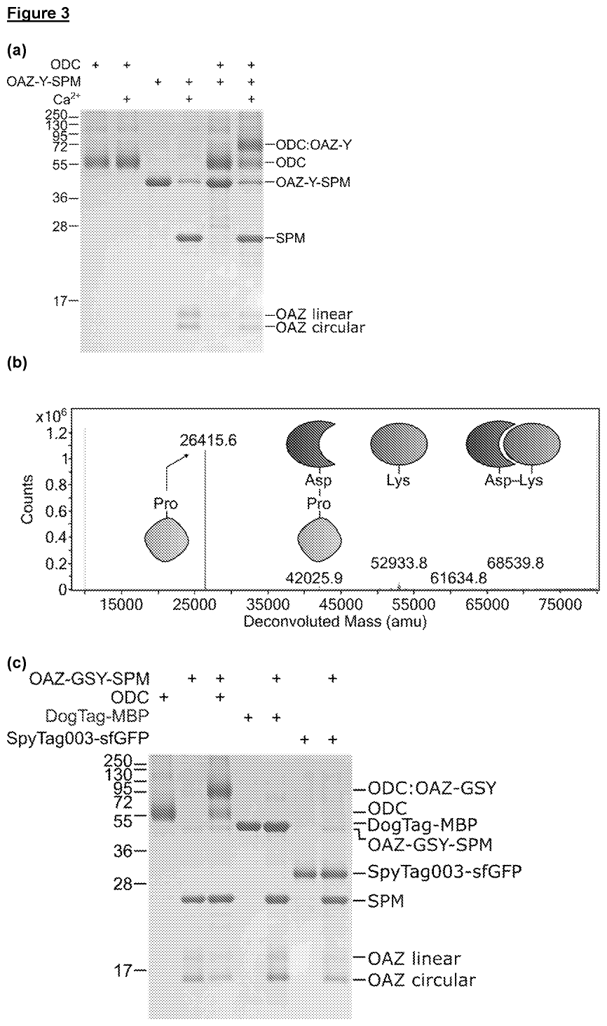

FIG. 3 shows (a) a photograph of an SDS-PAGE gel with Coomassie staining showing Ornithine Decarboxylase (ODC) reacted covalently with Ornithine decarboxylase antizyme (OAZ). ODC and OAZ-Y-SPM (with a Tyr before the SPM) were incubated at each 10 μM for 16 h with or without calcium, boiled in SDS loading buffer; (b) intact protein electrospray ionization MS confirms covalent coupling of OAZ-Y to ODC, with a loss of water (−18) indicating isopeptide formation and (c) a photograph of an SDS-PAGE gel with Coomassie staining showing specific reaction by NeissLock. OAZ-GSY-SPM was incubated overnight with each protein at 10 μM with the cognate partner ODC or non-cognate DogTag-MBP or SpyTag003-sfGFP. All lanes are in the presence of calcium. Samples were analyzed by SDS-PAGE with Coomassie staining.

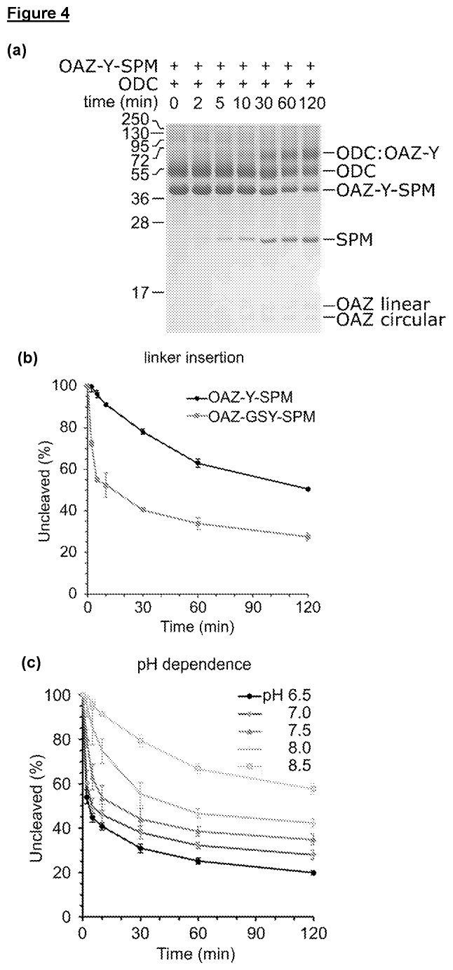

FIG. 4 shows (a) a photograph of an SDS-PAGE gel with Coomassie staining showing a time-course for OAZ-Y-SPM coupling. OAZ-Y-SPM was incubated with ODC for the indicated time in the presence of Ca²⁺; (b) a spacer increases cleavage efficiency. ODC was incubated with OAZ-Y-SPM or OAZ-GSY-SPM for the indicated time in the presence of Ca²⁺ and the extent of cleavage was determined by SDS-PAGE with Coomassie staining (mean of triplicate±1 s.d.; some error bars are too small to be visible); and (c) pH-dependence of cleavage. OAZ-GSY-SPM was incubated with Ca²⁺ for the indicated time at the indicated pH and cleavage of SPM was determined (mean of triplicate±1 s.d.).

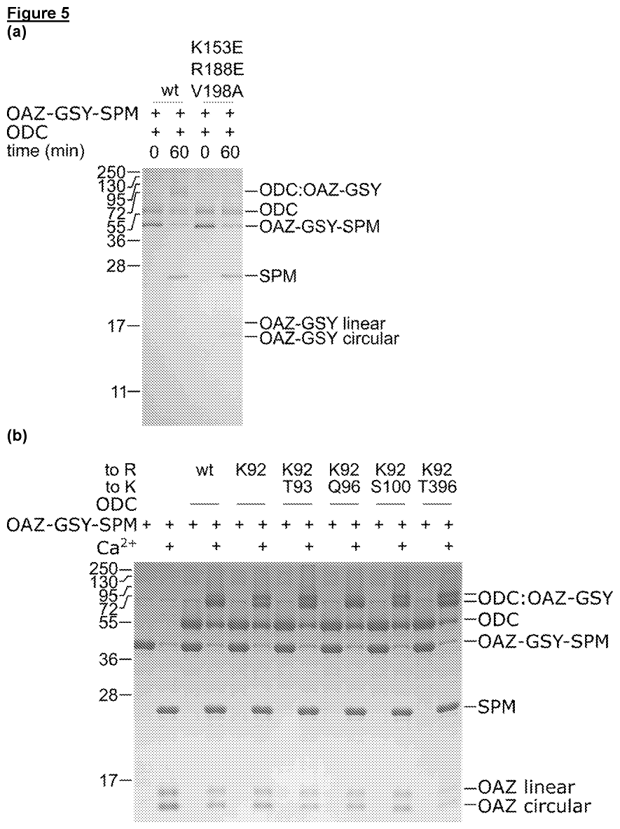

FIG. 5 shows (a) a photograph of an SDS-PAGE gel with Coomassie staining showing disruption of ODC/OAZ affinity blocked conjugation. OAZ-GSY-SPM or the non-binding OAZ-GSY-SPM was incubated with ODC along with Ca²⁺ with each protein at 0.5 μM for 0 or 60 min, before SDS-PAGE with Coomassie staining; and (b) a photograph of an SDS-PAGE gel with Coomassie staining showing different sites on ODC can react with OAZ. OAZ-GSY-SPM was incubated with the indicated ODC mutant overnight at 37° C. before SDS-PAGE with Coomassie staining.

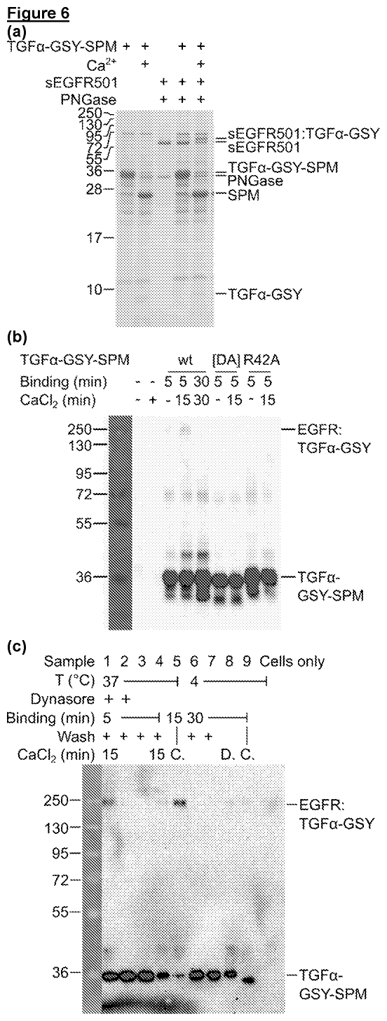

FIG. 6 shows (a) a photograph of an SDS-PAGE gel with Coomassie staining showing NeissLock reaction to soluble epidermal growth factor receptor (EGFR). TGFα-GSY-SPM was incubated with sEGFR with or without Ca²⁺ for 90 minutes at 37° C. Subsequently, samples were deglycosylated with PNGase F Kit (NEB), i.e. denatured with Glycoprotein Denaturing Buffer and digested at 37° C. with PNGase F before SDS-PAGE with Coomassie staining; (b) a photograph of a Western blot showing NeissLock conjugation to EGFR on cells. A431 cells were incubated with TGFα-GSY-SPM for 5 min at 37° C. or 30 min at 4° C. according to the indicated times. Samples were washed and optionally incubated with Ca²⁺ for 15 min at 37° C. or 30 min at 4° C. according to the indicated times. Non-processing TGFα-GSY-[DA]SPM or non-binding TGFα[R42A]-GSY-SPM controls were tested. Cells were lysed and Western blot was performed against Transforming Growth

Factor-alpha (TGFα); and (c) a photograph of a Western blot showing condition-dependence of reaction with EGFR. A431 cells were incubated with TGFα-GSY-SPM for varying times at different temperatures, before Western blot against TGFα. 1,2: Dynasore treated, 5 min binding to TGFα-GSY-SPM at 37° C., washed, with or without calcium for 15 min at 37° C. 3,4: As 2,1, respectively without prior dynasore treatment. 5: As 4, but cells were co-incubated with TGFα-GSY-SPM and calcium at the same time. 6,7: Cells were incubated with TGFα-GSY-SPM at 4° C. for 30 min, then washed, then without or with 30 min calcium incubation. 8: As 7, but cells were not washed before adding calcium. 9: As 5, but co-incubation for 30 min at 4 ° C. C: Control without TGFα-GSY-SPM.

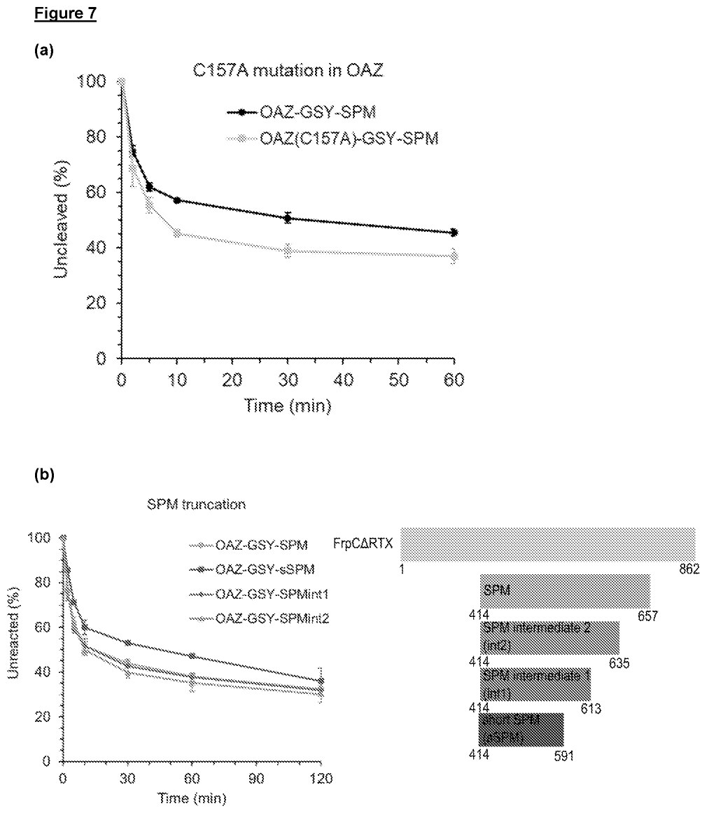

FIG. 7 shows (a) introduction of C157A in OAZ decreased protein aggregation and improved cleavage rate. OAZ-GSY-SPM or the C157A mutant was incubated with Ca²⁺ for the indicated time at 37° C. and cleavage was analyzed by SDS-PAGE with Coomassie staining; and (b) the effect of SPM truncations on cleavage. OAZ-GSY-SPM or various modifications (illustrated on right) were incubated at 37° C. with Ca²⁺ for the indicated time, before analysis of cleavage by SDS-PAGE with Coomassie staining. Data represent mean of triplicate±1 s.d. (some error bars are too small to be visible).

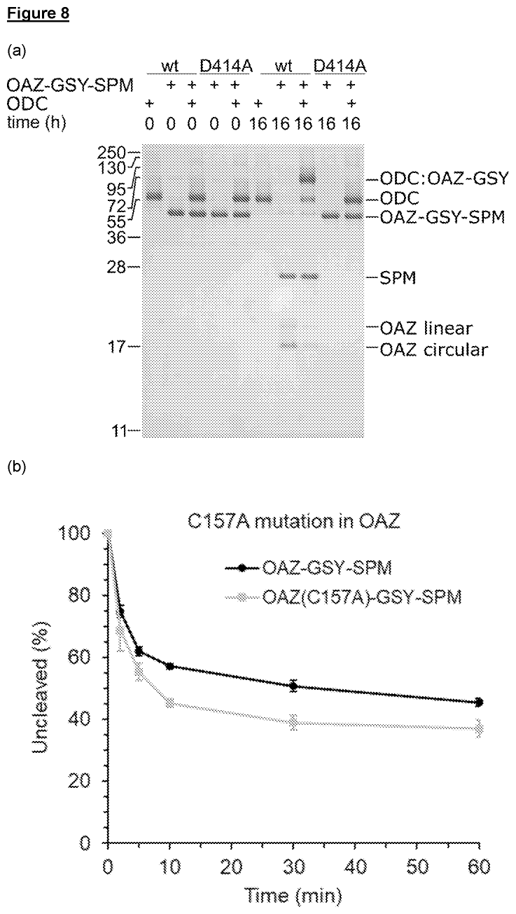

FIG. 8 shows a photograph of an SDS-PAGE gel with Coomassie staining showing the necessity of D414 in SPM for cleavage and coupling. ODC was incubated with OAZ-GSY-SPM with or without D414A mutation for the indicated time with Ca²⁺.

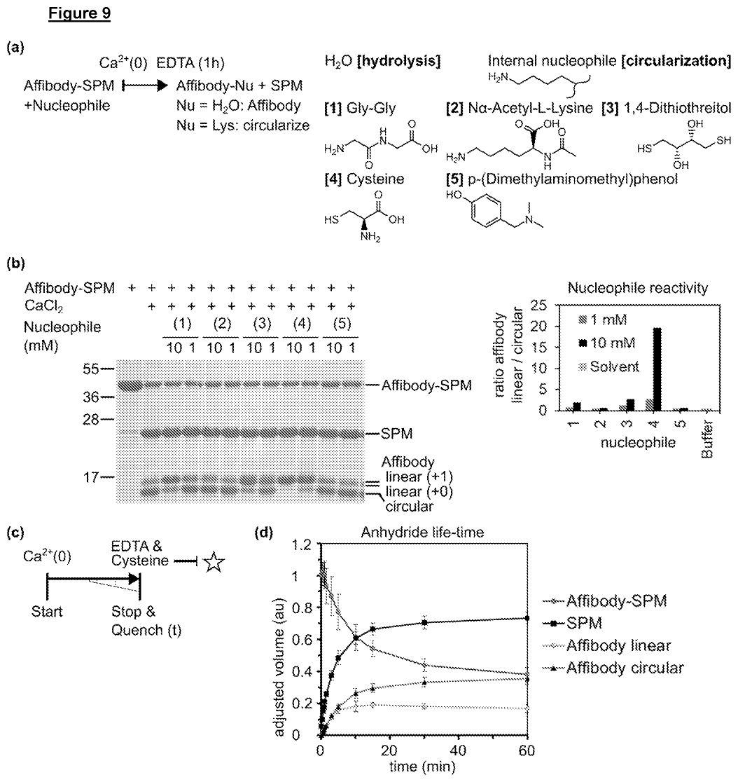

FIG. 9 shows the results of an investigation of aspartyl anhydride chemical reactivity. (a) After SPM activation by Ca²⁺, the released affibody features an aspartic anhydride. The anhydride then reacts with free nucleophiles or nucleophiles within the affibody (resulting in cyclization). Various nucleophiles were chosen: [1] N-terminal amine minic, [2] Lysine side-chain mimic, [3/4] thiols, and [5] Tyrosine side-chain mimic. [3] forms a labile thioester, whereas [4] may undergo S,N-acyl shift to yield an amide; (b) Quantification of nucleophile reaction with anhydride. Affibody-SPM was incubated with Ca²⁺ for 60 min at 37° C. in the presence of 1 or 10 mM of the indicated nucleophile. Products were analyzed by SDS-PAGE with Coomassie staining. Reaction with nucleophile in solution was quantified by the decrease in the level of cyclization. The ratio of linear to cyclized affibody is plotted at the right. (c, d) Anhydride lifetime. Generation of anhydride from affibody-SPM was initiated by adding Ca²⁺. At the indicated time-point, cleavage was stopped with EDTA and anhydrides were quenched with free cysteine. The abundance of each species was determined by SDS-PAGE with Coomassie staining. The different kinetics of SPM appearance and affibody cyclization are indicative of the life-time of the anhydride.

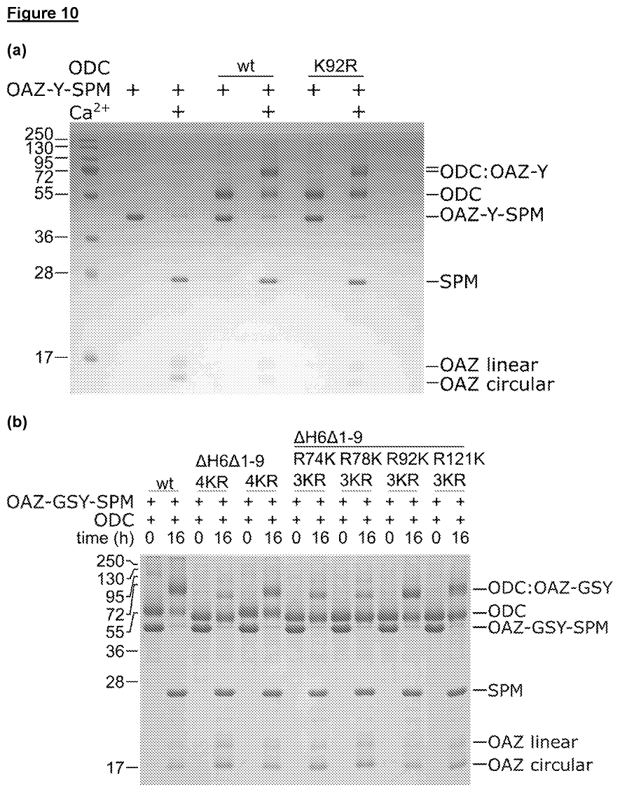

FIG. 10 shows the results of experiments to identify crosslinking sites for ODC reaction: (a) SDS-PAGE with Coomassie staining for OAZ-Y-SPM coupling to wt or K92R ODC. The position of K92 and K121 in the ODC/OAZ complex is shown (PDB 4ZGY); (b) Truncation of first 9 amino acids and removal of N-terminal His-tag (ΔH6Δ1-9), together with introduction of K92R, K12R, K74R and K78R (4KR) reduced conjugation of OAZ-GSY-SPM (SDS-PAGE with Coomassie staining). Re-insertion of the original N-terminus or re-introduction of K92 or K121 rescued coupling. Time where Ca²⁺ was present is indicated.

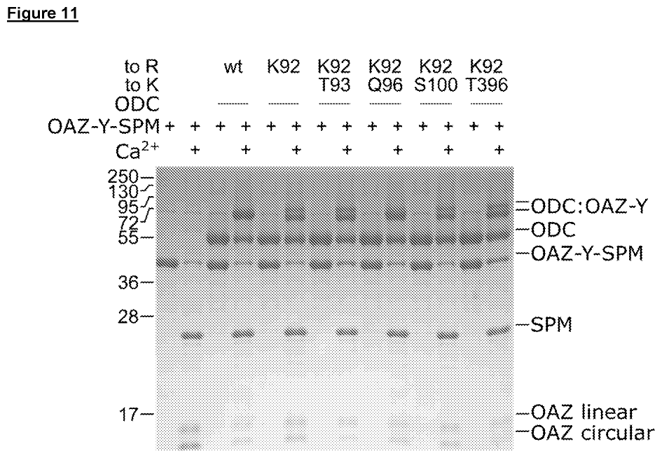

FIG. 11 shows a photograph of an SDS-PAGE gel with Coomassie staining showing changes in conjugation pattern for OAZ-Y-SPM to ODC K92R and ODC K92R double mutants. OAZ-Y-SPM was incubated with wt ODC or the indicated mutants with or without Ca2+.

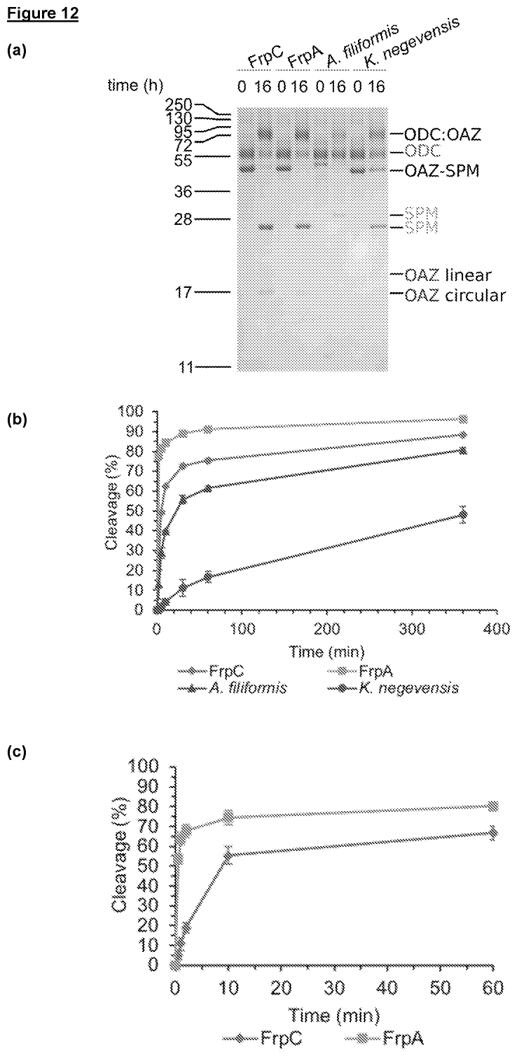

FIG. 12 shows cleavage and crosslinking activities of SPM homologues: (a) SPM homologues, including the −1 and −2 positions relative to the cleavable D-P bond, were fused to OAZ. The Coomasie-stained gel shows formation of cleavage and crosslinked products after 10 μM OAZ-SPM was incubated overnight with 10 μM ODC at 37° C., pH 7.4, and in the presence of 10 mM CaCl₂; (b) Different SPM homologues showed varying cleavage rates. Time course showing relative cleavage rates of different SPM homologues after incubation of 10 μM OAZ-SPM with 10 μM ODC at 37° C., pH 7.4, and with addition of 10 mM CaCl₂. Mean±1 s.d., n=3; and (c) FrpA cleaved more rapidly than FrpC even at 25° C. and with low calcium concentration. Time course showing cleavage rates of FrpA and FrpC. 10 μM OAZ-SPM was incubated with 10 μM ODC at 25° C., pH 7.4 and with addition of 1 mM CaCl₂. Mean±1 s.d., n=3.

### EXAMPLES

### Example 1—Characterisation of a Chimeric Protein Comprising a Self-Processing Module (SPM) From the FrpC Protein of Neisseria Meningitidis

To determine whether the cleavage efficiency of the SPM from the FrpC protein of Neisseria meningitidis could be maintained when coupled to other proteins, SPM (SEQ ID NO: 2) was fused to the unstructured SpyTag peptide and substituting each of the 20 amino acids in front of the reactive Asp-Pro was tested (SpyTag-X-SPM) (FIG. 1c). Protein constructs were all expressed in Escherichia coli and purified using Ni-NTA. Each purified chimeric protein was then incubated with 10 mM Ca²⁺ in the presence of 10 mM cysteine at 37° C. for 5, 15 or 60 min. The reaction was stopped by addition of EDTA and boiling in sodium dodecyl sulfate, ahead of SDS-PAGE with Coomassie staining (FIG. 1c). When X was D, G or P, there was minimal cleavage even after 60 min (FIG. 1d). H, Y, and W were most efficient at 60 min and Y is the residue here in the native FrpC. Therefore, Y was selected as the amino acid residue preceding the D-P dipeptide in the chimeric protein in further experiments for the development of “NeissLock”.

### Example 2—Semi-Automated Analysis of Protein Structures to Identify NeissLock Candidates

It was hypothesised that key features for the NeissLock strategy are likely to be the distance from the C-terminal anhydride to the nearest nucleophile on the binding protein, steric constraints so that the presence of SPM would disrupt complex formation, and the possibility of an own-goal (where a nucleophile on the target protein rather than the binding protein reacts with the anhydride) (FIG. 2a). A computational approach was developed to search the Protein Data Bank (PDB) to assess how complexes matched these criteria. The steps used by the computational approach are set out in FIG. 2b. First, a database with distances from the most distal resolved residue in a given polypeptide to nucleophilic residues in the same structure was generated from entries in the PDB. This database was then sorted and filtered, and structures were shortlisted after visualization and inspection in PyMOL (see Table 1 above). Due to promising structural characteristics, in combination with expression from E. coli, the complex between Ornithine Decarboxylase (ODC) and Antizyme (OAZ) (PDB 4ZGY) was selected as a model system. In addition, Epidermal Growth Factor Receptor/Transforming Growth Factor alpha (EGFR/TGFα, PDB ID 1MOX) was chosen for further study due to the biological importance of these proteins in cancer and cell survival.

### Example 3—Establishing the NeissLock Principle

In the ODC/OAZ crystal structure, the last resolved residue (E219) of OAZ is 3.5 A from K92 on ODC. Furthermore, E219 appeared to be sterically accessible and far from nucleophiles on OAZ itself. As further truncations in OAZ have previously been described, OAZ was truncated to E219 (hereafter referred to as “OAZ”) and Tyr was introduced as a spacer for SPM fusion (see above) to yield OAZ-Y-SPM as a chimeric protein comprising a binding polypeptide (i.e. a NeissLock-probe).

The boundaries of the SPM within FrpC are defined as 414-657. A stepwise truncation according to predicted secondary structure revealed that shortened forms of SPM (414-591, 414-613 and 414-635), while functional, were lower yielding and less pure than 414-657 after standard purification from E. coli expression. In addition, the shortened form of SPM (414-591) showed reduced cleavage rate (FIG. 7b). Thus, the full length “long” SPM (comprising amino acids 414-657 of FrpC, SEQ ID NO: 14) was selected for use in further experiments.

Upon addition of calcium, OAZ-Y-SPM undergoes self-processing to yield SPM and two OAZ species of differing mobility (FIG. 3a). Based on electrospray ionization-MS, these correspond to a linear OAZ species from hydrolysis and a cyclized species from self-reaction of a nucleophile on OAZ with its own anhydride. The formation of higher-molecular weight products indicative of self-conjugation of OAZ was observed in trace amounts (FIG. 3a). However, when ODC was mixed with OAZ-Y-SPM, no such higher-molecular weight products were observed. Instead OAZ nearly quantitatively conjugated to ODC (FIG. 3a). This covalent conjugation was validated by intact protein electrospray ionization MS. After OAZ-Y-SPM self-processing, masses corresponding to SPM (calculated: 26,414.80, observed: 26,415.61), OAZ-Y-SPM (calculated: 42,024.19, observed: 42,025.87), ODC (calculated: 52,929.42, observed: 52933.81) and ODC:OAZ-YD conjugate (calculated: 68,538.80, observed: 68,539.76) were identified (FIG. 3b). Mutation of D414 in SPM to alanine abolished calcium-induced cleavage of OAZ-GSY-SPM and covalent conjugation to ODC (FIG. 8), supporting the key role of this residue for reaction.

The parameters determining cleavage of the chimeric protein and conjugation of the binding and target polypeptide were explored using the ODC/OAZ model system. The OAZ-Y-SPM displayed reduced cleavage rate (FIG. 4a) compared to SpyTag-Y-SPM (FIG. 1c) or Affibody-Y-SPM (FIG. 9). Steric hindrance was proposed as the reason for reduced cleavage rate in SPM fusion proteins. Accordingly, a GS-linker was introduced into OAZ-Y-SPM to produce OAZ-GSY-SPM and its effect on cleavage rate and conjugation efficiency was tested. A significant increase in cleavage rate was observed in OAZ-GSY-SPM compared to OAZ-Y-SPM (FIG. 4b).

The pH-dependence of cleavage and conjugation was also tested using the ODC/OAZ model system. Since reaction is proposed to be principally from nucleophilic attack by the ε-amine of Lys, with a typical pKₐ of 10, it was important to test if the NeissLock approach was feasible at neutral pH (e.g. between pH 6.5 and 8.5). It was surprisingly found that cleavage was most efficient at pH 6.5 or 7.0 and but still readily occurred up to pH 8.5 (FIG. 4c). Similar to the cleavage rate, the rate of formation of the cross-linked product was highest at pH 6.5 and decreased with higher pH. However, significant changes in conjugation efficiency were not observed at different pH values within the tested range (calculated from the ratio of crosslinked product to cleavage product, SPM).

To investigate the specificity of NeissLock reaction, an AP-tag (Acceptor Peptide for site-specific biotinylation) was introduced to OAZ-GSY-SPM to enable SPR affinity measurements (AP-OAZ-GSY-SPM). Residue 175 in OAZ was changed from C to A (C175A) to produce AP-OAZ^{c175A}-GSY-SPM, in order to reduce aggregation (FIG. 7a). Conjugation of AP-OAZ^{c175A}-GSY-SPM to irrelevant (non-binding) proteins, maltose binding protein (MBP) or superfolder green fluorescent protein (sfGFP) was tested. At 10 μM concentration of both AP-OAZ^{c175A}-GSY-SPM and added protein, conjugation of 63% ODC to ODC:AP-OAZ^{c175A}-GSY-D was observed (FIG. 3c). For MBP, SDS-PAGE suggested trace levels of conjugation (1-2%), whereas no trace of conjugation to SpyTag003-sfGFP was identified (FIG. 3c).

The affinity-dependence of NeissLock was assessed. Two mutations reported to reduce binding in mouse OAZ/ODC (K153E and V198A) as well as a third mutation (charge inversion via R188E) were introduced into OAZ, to design the low affinity binder OAZ[K153E, R188E, V198A]-GSY-SPM. SPR was used to determine the KD of binding of AP-OAZ[K153E, R188E, V198A]-GSY-SPM to ODC and was found to be unmeasurable by SPR (indicating Kd>100 μM). For wild type AP-OAZ-GSY-SPM binding to ODC, a K_{d} of 0.12 μM was measured. Upon addition to ODC, no detectable cross-linked product was observed for AP-OAZ[K153E, R188E, V198A]-GSY-SPM after overnight incubation (FIG. 5a). This shows that the chimeric protein OAZ-Y-SPM and derivatives thereof may be used to selectively conjugate OAZ to ODC in an affinity-dependent manner, providing proof-of-concept for NeissLock conjugation.

### Example 4—Conjugation of OAZ to Other Nucleophiles in the Target Polypeptide, ODC

As discussed above, OAZ was identified as a suitable NeissLock probe (i.e. a binding polypeptide in the chimeric protein) based on the proximity of the distal resolved residue E219 to ODC K92 and it was hypothesized that crosslinking primarily occurred at ODC K92. Tryptic liquid chromatography-mass spectrometry/mass spectrometry (LC-MS/MS) was used to characterise OAZ-ODC conjugate produced from the OAZ-Y-SPM chimeric protein and crosslinked peptides at K92 were identified. When the OAZ-Y-SPM chimeric protein (NeissLock probe) was reacted with K92R ODC, it was surprisingly found that high amounts of covalent conjugation were observed (FIG. 5b and FIG. 10a). Optimized gel conditions were used to resolve at least two distinct conjugated species, suggesting that K92 is a primary crosslink site with alternative crosslink sites on ODC. The slower and faster migrating products of OAZ-Y-SPM conjugated to ODC K92R were subjected to tryptic LC-MS/MS. This resulted in the identification of another crosslink site at K121 in the slowly migrating product. The third crosslink site in either the slower or the faster migrating band was not identified by tryptic LC-MS/MS.

Comparison of OAZ-GSY-SPM to OAZ-Y-SPM resolved under optimized conditions revealed that OAZ-GSY-SPM showed traces of a distinct conjugation product even during conjugation with wild type ODC (FIG. 5b), whereas no such trace was observed for OAZ-Y-SPM (FIG. 11). This indicates that the GS spacer altered the availability of nucleophiles in the target polypeptide (ODC), potentially by introducing increased range and flexibility. To further explore the spatial requirements for crosslinking, attempts were made to rescue the wild type-like banding pattern for OAZ-Y-SPM or OAZ-GSY-SPM conjugation to ODC K92R by reintroducing lysine residues in proximity to K92R. Along the α-helix on which K92 is positioned, mutations T93K, Q96K or S100K were introduced into the ODC K92R background (FIG. 5b). Furthermore, ODC K92R T396K, which is opposite this α-helix, was tested (FIG. 5b). A wild type-like banding pattern was observed in ODC K92R Q96K (FIG. 4e), which has a similar orientation to K92—facing towards K121—and, especially accounting for the additional residues introduced after E219, i.e. -YD or -GSYD, is likely at a comparable distance to the aspartic anhydride.

Finally, additional crosslinking sites on ODC were located by rational mutagenesis. From tryptic LC-MS/MS, K92 and K121 were already identified as crosslinking sites. The faster migration of one of the product bands indicated that the crosslinking products would be less branched than for crosslinking to ODC K121, i.e. closer to the terminus. First, lysines in proximity to OAZ E219 were mutated to make ODC ‘4KR’ (ODC K92R K121R K74R K78R) and ODC ‘8KR’ (ODC 4KR with additional K141, K69, K148 and K150). Compared to wild-type ODC, conjugation of OAZ-GSY-SPM to ODC 4KR or ODC 8KR showed similarly reduced efficiency, however, significant amounts of product formation were still observed. It was hypothesised that the unresolved N-terminal region of ODC—which further harbours flexible tags—could be another crosslinking site, especially considering the good reactivity of the N-terminal amine (FIG. 9). Although the proximal resolved residue of ODC in the 4ZGY structure faces outwards, far away from the ODC/OAZ interface, alignment of a structure of the ODC homodimer (PDB ID 1D7K) indicated that the N-terminus of ODC might loop back towards the binding interface.

Accordingly, the mutations in ODC 4KR were combined with removal of the N-terminal His-tag as well as further truncation of unresolved or flexible residues based on PDB 1D7K to make ‘ΔH6Δ1-9 4KR’. Although removal of the His-tag alone did not appear to significantly reduce conjugation efficiency, conjugation of OAZ-GSY-SPM to ΔH6Δ1-9 4KR yielded only low amounts of conjugation. The amount of crosslinked product was 6.7% that of wild type ODC (FIG. 10b). Subsequent reintroduction of R74K and R78K into ODC 4KR appeared to rescue none to small amounts of conjugation (FIG. 10b), consistent with the observations made for spatial preference in ODC K92R Q96K compared to ODC K92R T93K/S100K (FIG. 5b). However, reintroduction of either R92K or R121K rescued high levels of conjugation, notably yielding a slow migrating species as the main product for ODC 4KR R121K, consistent with previous observations. These observations confirm a third major crosslinking site within the N-terminal region, likely at the N-terminal amine as this region contains no lysines. Overall, conjugation appears to be most efficient for K92 and, according to the ratio of slow and fast migrating bands in ODC K92R (FIG. 5b), similarly efficient for K121 and the N-terminal amine, wherein these ratios are influenced by linker length (FIG. 5b and FIG. 11). In summary, the OAZ anhydride conjugates efficiently with multiple different crosslinking sites on ODC.

To understand the solution behaviour of OAZ-GSY-SPM, size exclusion chromatography with multi-angle light scattering (SEC-MALS) was performed. This analysis gave a close correspondence between the predicted and observed M_{W} for a monomeric protein.

### Example 5—Use of a Chimeric Protein (TGFα-GSY-SPM) to Conjugate a Polypeptide to Cells

The TGFα/EGFR complex was identified as a promising candidate for use in the method of the invention. This was validated by testing conjugation of TGFα-GSY-SPM to the soluble ectodomain fragment of EGFR, sEGFR501 in vitro. The complex glycosylation of sEGFR501 expressed in 293Expi cells led to heterogeneous gel mobility. Therefore this construct was expressed with the mannosidase inhibitor kifunensine and treated with PNGase F before resolving it on SDS-PAGE, which resulted in a single sharp band. Co-incubation of 10 μM sEGFR with 100 μM TGFα-GSY-SPM in the presence of Ca²⁺ led to the formation of a new species, a covalent complex between sEGFR and TGFα, which is not present from autoproteolysis of TGFα-GSY-SPM alone (FIG. 6a). Under these conditions, ˜50% of sEGFR was conjugated.

To test cellular interaction of SPM fusion, the interaction of TGFα-GSY-SPM at the mammalian cell surface was assessed. The A431 cell line, which displays high levels of EGFR, was used. MCF-7 was used as a negative control since it has low levels of EGFR. AlexaFluor-488 conjugated anti-EGFR affibody was used as a positive control. His₆-TGFα-SPM detected with anti-His-phycoerythrin (PE) resulted in clear visualization of A431 cellular membranes, which was not the case for MCF-7, supporting specific receptor binding. Covalent reaction of TGFα-GSY-SPM to EGFR on cells was then tested. A431 cells incubated with TGFα-GSY-SPM showed conjugation of TGFαto EGFR as determined by Western blot (FIG. 6b). Importantly, incubation with either TGFα-GSY-[DA]-SPM (non-cleaving) or TGFα[R42A]-GSY-SPM, a low-binding mutant of TGFα, blocked reaction, indicating that conjugation was dependent on both SPM-processing and EGFR-binding (FIG. 6b). Subsequent testing of different cleavage conditions showed that both co-incubation of TGFα-GSY-SPM with calcium as well as inhibition of endocytosis with dynasore further improved coupling yield (FIG. 6c).

### Example 6—Characterising Other Self-Processing Modules and Their Use in the Chimeric Protein

To verify the utility of other SPMs in the methods and uses of the invention, SPMs with homology to the SPM from FrpC protein from Neisseria meningitidis (SEQ ID NO: 2) were identified. In particular, an SPM was identified in: the FrpA protein from Neisseria meningitidis (SEQ ID NO: 1), which shows 98.37% sequence identity to SEQ ID NO: 2; the haemolysin-type calcium binding protein related domain-containing protein from Alysiella filiformis (SEQ ID NO: 3), which shows 71.95% sequence identity to SEQ ID NO: 2; and the bifunctional haemolysin/adenylate cyclase precursor protein from Kingella negevensis (SEQ ID NO: 4), which shows 60.41% sequence identity to SEQ ID NO: 2.

Each of the SPMs was used to produce a chimeric protein containing a domain (i) sequence containing AP-GSS-His6-OAZ (SEQ ID NO: 13); a linker domain comprising GVY, GIV or GGY, and the SPM sequence set out above. The sequences of the chimeric proteins are set out in SEQ ID NOs: 9-12 (i.e. comprising SEQ ID NOs: 1-4, respectively).

The chimeric proteins were assessed for their ability to promote the proximity-dependent conjugation of OAZ to ODC as described in Example 3. As shown in FIG. 12a, all of the chimeric proteins were able to promote the proximity-dependent conjugation of OAZ to ODC. Moreover, it was surprisingly determined that the SPM from FrpA (SEQ ID NO: 1) displayed a substantially faster rate of autoproteolytic cleavage and a higher yield of cleavage compared to the other SPMs (FIGS. 12b and 12c). Notably, SEQ ID NO: 1 differs from SEQ ID NO: 2 at positions 17 (A vs T), 23 (A vs S), 28 (R vs T) and 30 (Q vs N) (using the numbering of SEQ ID NOs: 1 and 2). It is hypothesised that one or all of these differences results in the improved activity of the SPM from the FrpA protein.

### METHODS

### Plasmids and Cloning

For cloning of constructs, Q5 High-Fidelity Polymerase (NEB) or KOD Hot Start DNA Polymerase (EMD Millipore) was used for PCR followed by Gibson assembly. Residue numbers for SPM derive from FrpC of N. meningitidis serogroup B (strain MC58) (UniProt Q9JYV5). The SPM sequence was based on residues 414-657 of FrpC. SpyTag-A-SPM has the following organization: N-terminal (M)GSS-linker, His₆-tag, SSG-linker, thrombin cleavage site, Ndel restriction site, G-spacer, SpyTag, alanine, SPM, GSG-linker, C-tag. Residue numbers for OAZ and ODC were based on the crystal structure of the OAZ:ODC complex (PDB 4zgy). Residues 95-219 of human OAZ (UniProt P54368) were used for pET28a-His₆-OAZ-SPM-Ctag. The truncation of OAZ1 corresponds to the region modelled in

PDB 4zgy. pET28a-His₆-OAZ-SPM-Ctag has the following organization: N-terminal (M)GSS-linker, His₆-tag, OAZ, SPM, GSG-linker, C-tag. Human ODC1 (UniProt P11926) was cloned into pET28a-His₆-ODC-Ctag to give the following organization: N-terminal (M)GSS-linker, His₆tag, SSG-linker, ODC1, GSG-linker, C-tag. pET28a-TGFα-GSY-SPM-His₆-Ctag includes mature TGFαsequence that was taken from residues 40-89 of human protransforming growth factor alpha (UniProt P01135). His₆-TGFα-SPM has the following organization: N-terminal (M)GSS-linker, His₆-tag, SSG-linker, TGFα, SPM, GSG-linker, C-tag. DNA primers and gene fragments codon optimized for E. coli expression were ordered from Integrated DNA Technologies before cloning into the pET28a backbone. All constructs were validated by Sanger sequencing.

### Mammalian Protein Expression

Expression of the ectodomain of human EGFR was carried out using pENTR4-sEGFR501-His₆ that has the organization: tissue plasminogen activator (tPA) secretion leader sequence, soluble fragment of extracellular domain of human EGFR (UniProt P00533, residues 25-525), GSGESG (SEQ ID NO:15), His₆s. pENTR4-sEGFR501-His₆was transfected into the Expi293 Expression System (ThermoFisher) using the ExpiFectamine 293 Transfection Kit (ThermoFisher).

Secreted sEGFR501 was recovered from the cell supernatant using Ni-NTA affinity purification.

### Database Search for Model Protein Complex

To identify candidate complexes for covalent fusion by C-terminal activation, protein structures were screened for the distance of the C-terminal resolved residue to Lys ε-amino groups (CTε). First, protein structures were retrieved from the worldwide protein data bank (wwPDB, www.wwpdb.org). Initial analysis was performed using the programming language Python (Python Software Foundation, www.python.org); in particular, the Biopython PDB module was used to interpret structural data. A set of protein structures was pre-selected based on inter- and intra-chain CTε, chain count, and other metadata. Preselected structures were visually inspected in PyMOL (version 2.0) and a final selection was made, taking into account the biological relevance of the complex and experimental data such as ease of purification and complex K_{d}.

### Bacterial Protein Expression and Purification

For pET28a-His₆-OAZ-SPM-Ctag, pET28-His₆-ODC1-Ctag or related plasmids, the plasmids were transformed into chemically-competent E. coli BL21 (DE3) RI PL (Agilent Technologies). Cells were then plated on LB agar with 50 pg/mL kanamycin and incubated overnight at 37° C. Single colonies were picked to inoculate 11 mL of LB with 50 μg/mL kanamycin and 34 μg/mL chloramphenicol before 16-20 hours of incubation at 37° C. with shaking at 200 rpm. 10 mL of the overnight culture was used to inoculate 1 L of LB with 50 μg/mL kanamycin and 34 μg/mL chloramphenicol in a baffled flask. Cultures were incubated at 37° C. with shaking at 200 rpm until OD₆₀₀ reached ˜0.6, upon which cultures were induced using with 0.42 mM isopropyl β-D-1-thiogalactopyranoside (IPTG) (Fluorochem, UK) and incubated at 25° C. with shaking at 200 rpm for 16-18 h. Cells were harvested from the culture medium with a JLA8.1 rotor at 4° C., washed and pelleted. Cell pellets were immediately processed or stored at −80° C. until further use. For constructs containing TGFα-SPM and variants thereof, the same protocol was used except that protein was induced at 18° C. instead of 25° C. For His₆-TGFα-SPM, the protein was induced from the Rosetta-Gami 2 (DE3) strain instead of BL21 (DE3) RIPL.

### ODC and OAZ-SPM Protein Purification

For variants of ODC and OAZ-SPM, cells were harvested and lysed by sonication in lysis buffer [30 mM Tris-HCl, 200 mM NaCl, 5% (v/v) Glycerol, 15 mM imidazole, pH 7.5] supplemented with mixed protease inhibitors (cOmplete mini EDTA-free protease inhibitor cocktail, Roche), 1 mM phenylmethylsulfonyl fluoride (PMSF), 1 mg/mL lysozyme (Sigma-Aldrich), 2 U/mL benzonase (Sigma-Aldrich) and 5 mM 2-Mercaptoethanol (Sigma-Aldrich). While kept on ice, the lysate was sonicated thrice for 1 min at 50% duty cycle with 1 min rest period in between. The cell lysate was then centrifuged at 16,900 g for 10-20 min at 4° C. The clarified lysate was then added to Ni-NTA resin (Qiagen). After addition to a Polyprep gravity column, the Ni-NTA resin was washed twice with 5 packed resin volumes of Ni-NTA buffer (50 mM Tris-HCl, 300 mM NaCl, pH 7.8) with 10 mM imidazole and 5 mM 2-Mercaptoethanol (Sigma-Aldrich). This was followed by another two washes with 5 packed resin volumes of Ni-NTA buffer with 30 mM imidazole and 5 mM 2-Mercaptoethanol (Sigma-Aldrich). The protein was eluted from the Ni-NTA resin using Ni-NTA buffer with 200 mM imidazole and 5 mM 2-Mercaptoethanol (Sigma-Aldrich). The protein was concentrated using a Vivaspin centrifugal concentrator with 10 or 30 kDa cut-off (GE Healthcare) before loading onto a pre-equilibrated HiLoad 16/600 Superdex 200 pg size exclusion chromatography column (GE Healthcare) connected to an AKTA Pure 25 (GE Healthcare) fast protein liquid chromatography (FPLC) machine at 4° C. 50 mM HEPES, 150 mM NaCl, 2 mM TCEP, pH 7.4 buffer was used for gel filtration. An additional 0.02 mM pyridoxal phosphate (PLP) was added to the gel-filtration buffer when purifying ODC. Fractions were collected according to the A₂₈₀ peak and verified by SDS-PAGE, before another round of concentration using Vivaspin centrifugal concentrator with 10 or 30 kDa cut-off (GE Healthcare). For ODC variants without an N-terminal His-tag, the clarified lysate was added to CaptureSelect™ C-tagXL Affinity Matrix (ThermoFisher) instead of Ni-NTA. After addition to a Polyprep gravity column, the resin was washed four times with 5 packed resin volumes of wash buffer (20 mM Tris-HCl, 5 mM 2-Mercaptoethanol, pH 7.4). The protein was eluted from the C-tagXL resin using 50 mM HEPES, 5 mM 2-Mercaptoethanol and 2M MgCl₂, pH 7.8.

### Protein Analysis

Protein concentrations were estimated using a NanoDrop spectrophotometer, with extinction coefficients estimated using the ExPASy server. SDS-PAGE was done using 10%, 16% or 18% polyacrylamide gels in an XCell SureLock system (ThermoFisher) run at 180V or 200V. SDS-PAGE gels were stained using InstantBlue (Expedeon) and destained with water before imaging with a ChemiDoc XRS imager. Quantification was carried out using Image Lab software (version 5.2.1).

### Cleavage and Coupling Assays

Reactions were carried out in the reaction buffer (50 mM HEPES, 150 mM NaCl, 2 mM TCEP, pH 7.4) at 37° C. When measuring the pH-dependence of the reaction, an additional 50 mM 2-(N-morpholino)ethanesulfonic acid (MES) was added for proper buffering over the pH range tested. For reactions analyzing the effect of the −1 position on cleavage rate, 10 μM of SpyTag-X-SPM was used. For reactions for analysing the speed and pH-dependence of coupling, OAZ-SPM was reacted with ODC at a 1:1 ratio with each protein at 10 μM or at the indicated concentrations. The cleavage of SPM was induced by addition of the HEPES reaction buffer, pre-equilibrated to 37° C., containing calcium chloride at a final concentration of 10 mM. After the indicated time, the reaction was stopped by addition of 5×SDS-loading buffer [0.19 M Tris-HCl pH 6.8, 20% (v/v) glycerol, 100 μM bromophenol blue, 0.19 M SDS] containing EDTA added to a final concentration of 15 mM in the reaction mixture. Protein samples were then heated on a Bio-Rad C1000 thermal cycler at 95° C. for 3 min. For time courses, the 0 h time point was taken by addition of the stop buffer to the reaction before addition of the start buffer. Finally, cleavage and coupling reactions were analyzed by gel densitometry of 10%, 16% or 18% polyacrylamide gels. The percentage cleavage of SPM was determined from the reduction in intensity of SpyTag-X-SPM or OAZ-SPM from the 0 h time point.

### Anhydride Reactivity Test

20 μM Affibody-SPM was incubated with 10 mM CaCl₂ in 50 mM HEPES, 150 mM NaCl, pH 7.4 (HBS) with 1 mM or 10 mM of the indicated nucleophiles at 37° C. for 1 h, before inhibiting the reaction with 75 mM EDTA in 5×SDS loading buffer. Samples were resolved on 18% SDS-PAGE without prior boiling. For anhydride lifetime tests, 7.5 μM Affibody-SPM was incubated for the indicated amount of time with 10 mM CaCl₂ in 50 mM HEPES, 150 mM NaCl, pH 7.4. Samples were then quenched with 5 μL 100 mM EDTA and 100 mM Cysteine in HBS. Samples were boiled in SDS loading buffer before resolving on SDS-PAGE.

### SEC-MALS

OAZ-SPM was prepared at 2 mg/mL in 100 μL of buffer containing 50 mM HEPES, 150 mM NaCl, 2 mM TCEP, 0.02 mM PLP, pH 7.4 before injection into a Superdex 200 HR 10/30 column (GE Healthcare) connected to a Shimadzu HPLC system with an attached Wyatt Dawn HELEOS-II 8-angle light scattering detector and Wyatt Optilab rEX refractive index monitor. SEC-MALS was carried out at room temperature with 50 mM HEPES, 150 mM NaCl 2 mM TCEP, 0.02 mM PLP, pH 7.4 running buffer.

### Surface Plasmon Resonance

Surface plasmon resonance was carried out using a Biacore T200 (GE Healthcare). AP-OAZ-GSY-SPM was biotinylated using GST-BirA. Biotinylated AP-OAZ-GSY-SPM was immobilized onto the sensor chip using the Biotin CAPture reagent from the Biotin CAPture Kit, Series S (GE Healthcare) and following each run, the chip was regenerated using the provided solutions, following the manufacturer's protocol. Serial dilutions of ODC were tested when measuring the K_{d} of OAZ binding to ODC. 1.25 μM ODC was diluted down to 78.1 nM for wild-type OAZ and for binding mutants of OAZ, 97 μM of ODC was diluted down to 1.51 μM.

### Mass Spectrometry

For intact protein mass spectrometry, a RapidFire 365 platform (Agilent) comprising a jet-stream electrospray ionization source coupled to a 6550 Accurate-Mass Quadrupole Time-of-Flight (Q-TOF) (Agilent) detector was used. With the RapidFire platform, protein samples prepared at 10 μM in 70 μL were acidified to 1% (v/v) formic acid before aspiration under vacuum for 0.3 s and loading onto a C4 solid-phase extraction cartridge. Washes using 0.1% (v/v) formic acid in water was carried out for 5.5 s before sample elution onto the Q-TOF detector for 5.5 s.

### Tryptic LC-MS/MS

Conjugated OAZ-Y-SPM/ODC or OAZ-Y-SPM/ODC K92R were resolved on 18% SDS-PAGE at 180 V for 100 min to separate different conjugate species. Bands were cut from the gel, in particular higher and lower conjugate bands, and submitted to the Oxford Biochemistry Proteomics facility for further processing.

### Cell Staining with TGFα-SPM

A431 and MCF-7 were cultured in Dulbecco's Modified Eagle Medium supplemented with 10% fetal bovine serum, 1% penicillin, 1% streptomycin, and 1% GlutaMAX at 37° C., 5% CO₂. Before cell staining, A431 and MCF-7 was seeded onto glass-bottom petri dishes. The glass dishes were transferred to 4° C. to prevent receptor internalization, the medium was removed, and cells were washed twice with 1 mL PBS +5 mM MgCl₂ (PBS-M). Then, cells were incubated with PBS-M with 1% (w/v) bovine serum albumin (BSA) and 1.5 μM anti-EGFR-Affibody conjugated to AlexaFluor-488 or 3 μM His₆-TGFα-SPM (from Rosetta-Gami 2) as indicated. After 30 min, cells were washed twice with PBS-M +1% BSA. Samples not incubated with affibody were incubated with 450 μL Anti-His-Phycoerythrin at 1:200 in PBS-M +1% BSA; affibody samples were incubated with only PBS-M +1% BSA instead. After 15 min, cells were washed twice and then covered with 1 mL PBS-M. Samples were imaged with a DV core inverted microscope (Micron Oxford), using a FITC (green false colour) or TRITC filter (red false colour).

### Cell Conjugation with TGFα-GSY-SPM

A431 cells were seeded into 25 cm² flasks and grown overnight. Before cell conjugation, cells were starved in Dulbecco's Modified Eagle Medium. For cell conjugation, TG Fα-GSY-SPM, TGFα-GSY-[DA] SPM or TGFα[R42A]-GSY-SPM diluted in HEPES-buffered saline (50 mM HEPES, 150 mM NaCl, pH 7.4) supplemented with 5 mM MgCl₂ (HBS-M) were added to cells. Cells were either incubated for the indicated time at indicated temperature before washing with HBS-M. Subsequently, 2 mM CaCl₂ diluted in HBS-M was added to the cells. Alternatively, CaCl2 diluted in HBS-M was added immediately after addition to the protein solution without washing (co-incubation) or added after the indicated amount of time without washing (directly). After protein conjugation, cells were placed on ice and washed with HBS-M. Optionally, cell flasks were frozen at −80° C. before further processing. Cells were lysed by addition of hot SDS lysis buffer (1% SDS in 10 mM Tris-HCl, 1 mM EDTA, pH 8.0), followed by sonication, heating and centrifugation.

### Western Blot

Cell lysates were diluted in reducing SDS loading buffer and resolved on SDS-PAGE as described above. Proteins were transferred overnight at 30 V, 4° C. to methanol-activated Polyvinylidene fluoride (PVDF) membrane in transfer buffer (7.2 g/L glycine, 1.44 g/L Tris base in 20% methanol). Membranes were blocked with 5% (w/v) skim milk in PBS pH 7.4 with 0.05% (v/v) Tween-20 (PBS-T). Subsequently, membranes were incubated with primary antibodies at 1:1000 dilution in 5% (w/v) skim milk in PBS-T, i.e. mouse anti-TGFα (MF9, Novus Biologicals) or mouse anti-EGFR (LA22, Merck). Membranes were washed 3-4 times with PBS-T before addition of secondary goat anti-mouse horseradish peroxidase HRP antibody (Sigma-Aldrich A4416) at 1:5000 dilution in 5% (w/v) skim milk with PBS-T. After additional washes with PBS-T, membranes were incubated with SuperSignal™ West Pico PLUS Chemiluminescent Substrate before measuring chemiluminescence on a ChemiDoc XRS imager.

### Graphics/ Structure Visualization

The structure of OAZ/ODC was obtained from PDB 4zgy and TGFα/EGFR from PDB 1mox, respectively. Structures were visualized using PyMOL (version 2.0). Figures were prepared using the FIJI distribution of ImageJ and the open-source graphics editor inkscape (inkscape.org).

1. Use of a chimeric protein to generate an anhydride group on a polypeptide for the formation of a covalent bond, wherein the chimeric protein comprises:
(i) a domain comprising the polypeptide; and
(ii) a domain comprising a self-processing module that contains an N-terminal dipeptide of aspartate or glutamate and proline (D/E-P),
wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue in the self-processing module to release the polypeptide and generate the anhydride group on the aspartate or glutamate residue.

2. The use of claim 1 further comprising using the anhydride group on the polypeptide to: (i) form an intramolecular covalent bond in the polypeptide; or (ii) conjugate the polypeptide to a second polypeptide via a covalent bond.

3. A method of producing an anhydride group on a polypeptide for use in directing the formation of a covalent bond comprising:
(a) providing a chimeric protein comprising:
(i) a domain comprising the polypeptide; and
(ii) a domain comprising a self-processing module that contains an N-terminal dipeptide of aspartate or glutamate and proline (D/E-P),
wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions;
(b) inducing the self-processing module to cleave the peptide bond between the proline residue and the aspartate or glutamate residue to release the polypeptide and generate the anhydride group on the aspartate or glutamate residue,
thereby producing a polypeptide comprising an anhydride group.

4. The method of claim 3 further comprising a step of isolating the polypeptide comprising an anhydride group and/or storing the polypeptide comprising an anhydride group under conditions in which the anhydride group is stable.

5. The method of claim 3, being a method of forming an intramolecular covalent bond in a polypeptide (e.g. a method of cyclizing a polypeptide) comprising:
(a) providing a chimeric protein comprising:
(i) a domain comprising the polypeptide; and
(ii) a domain comprising a self-processing module that contains an N-terminal dipeptide of aspartate or glutamate and proline (D/E-P),
wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions; and
(b) inducing the self-processing module to cleave the peptide bond between the proline residue and the aspartate or glutamate residue to release the polypeptide and generate an anhydride group on the aspartate or glutamate residue that reacts with a functional group in the polypeptide to form a covalent bond,
thereby forming an intramolecular covalent bond in the polypeptide (e.g. thereby cyclizing the polypeptide).

6. Use of claim 1 or 2, being the use of a chimeric protein to conjugate a first polypeptide to a second polypeptide via an isopeptide bond, wherein the chimeric protein comprises:
(i) a domain comprising the first polypeptide; and
(ii) a domain comprising a self-processing module that contains an N-terminal dipeptide of aspartate or glutamate and proline (D/E-P),
wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue in the self-processing module to release the first polypeptide and generate an anhydride group on the aspartate or glutamate residue at the C-terminus of the first polypeptide that reacts with a functional group on the second polypeptide to form the covalent bond.

7. The use of claim 6, wherein the second polypeptide binds non-covalently to the chimeric polypeptide via an interaction with the domain comprising the first polypeptide.

8. The method of claim 3, being a method of conjugating a first polypeptide to a second polypeptide via a covalent bond comprising:
(a) providing a chimeric protein comprising:
(i) a domain comprising the first polypeptide; and
(ii) a domain comprising a self-processing module that contains an N-terminal dipeptide of aspartate or glutamate and proline (D/E-P),
wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions;
(b) contacting the chimeric protein of (a) with the second polypeptide, wherein the second polypeptide binds non-covalently to (i);
(c) inducing the self-processing module to cleave the peptide bond between the proline residue and the aspartate or glutamate residue to release the first polypeptide and generate an anhydride group on the aspartate or glutamate residue that reacts with a functional group on the second polypeptide to form the covalent bond, thereby conjugating the first and second polypeptides.

9. The use or method of any preceding claim, wherein the covalent bond is an amide bond.

10. The use or method of any preceding claim, wherein the functional group is an amine.

11. The use of any one of claim 6, 7, 9 or 10, or the method of any one of claims 8 to 10, wherein the second polypeptide is attached to the surface of a cell or is in the extracellular matrix.

12. The use or method of claim 11, wherein the cell is located in a subject or the extracellular matrix is located in an organ and/or subject.

13. The use of any one of claim 6, 7, 9 or 10 or method of any one of claims 8 to 10, wherein the second polypeptide is attached to an exosome, virus, virus-like particle, nanoparticle or solid support.

14. The use or method of any preceding claim, wherein the self-processing module comprises:
(1) an amino acid sequence as set forth in SEQ ID NO: 1;
(2) a portion of (1) comprising an amino acid sequence as set forth in SEQ ID NO: 5;
(3) an amino acid sequence with at least 80% sequence identity to an amino acid sequence as set forth in SEQ ID NO: 1 or 2; or
(4) a portion of (3) comprising an amino acid sequence with at least 80% sequence identity to an amino acid sequence as set forth in SEQ ID NO: 5 or 6,
wherein the amino acid sequence comprises aspartate or glutamate at position 1, proline at position 2 and one or more of the following:
1) alanine at position 17;
2) alanine at position 23;
3) arginine at position 28;
4) glutamine at position 30;
and wherein the self-processing module cleaves the peptide bond between the first and second amino acids of the domain comprising a self-processing module under suitable conditions.

15. The use or method of any preceding claim, wherein the self-processing module comprises:
(1) an amino acid sequence as set forth in SEQ ID NO: 1;
(2) a portion of (1) comprising an amino acid sequence as set forth in SEQ ID NO: 5;
(3) an amino acid sequence with at least 99% sequence identity to an amino acid sequence as set forth in SEQ ID NO: 1; or
(4) a portion of (3) comprising an amino acid sequence with at least 99% sequence identity to an amino acid sequence as set forth in SEQ ID NO: 5,
wherein the amino acid sequence comprises aspartate or glutamate at position 1 and proline at position 2;
and wherein the self-processing module cleaves the peptide bond between the first and second amino acids of the domain comprising a self-processing module under suitable conditions.

16. A composition comprising: (i) a polypeptide having an anhydride group on a C-terminal aspartate or glutamate residue, wherein the aspartate or glutamate residue in the polypeptide is not present at the equivalent position in the amino acid sequence of the corresponding endogenous polypeptide or portion thereof; and (ii) a solvent that prevents hydrolysis or reaction of the anhydride group.

17. A polypeptide (e.g. a cyclized polypeptide) comprising an intramolecular covalent bond formed between an aspartate or glutamate residue and a functional group (e.g. an amine on a lysine residue or at the N-terminus), wherein:
(i) the aspartate or glutamate residue in the polypeptide is not present in the amino acid sequence of the corresponding endogenous polypeptide or portion thereof; and
(ii) the functional group in the polypeptide is present at an equivalent position in the corresponding endogenous polypeptide or portion thereof.

18. A product comprising a first polypeptide conjugated to a second polypeptide via a covalent bond between an aspartate or glutamate residue in the first polypeptide and a functional group in the second polypeptide, wherein:
(i) the aspartate or glutamate residue in the first polypeptide is not present at the equivalent position in the amino acid sequence of the corresponding endogenous polypeptide or portion thereof; and
(ii) the functional group in the second polypeptide is present at the equivalent position in the amino acid sequence of the corresponding endogenous polypeptide.

19. The product of claim 18, wherein:
(i) the first polypeptide comprises an amino acid sequence that corresponds to the amino acid sequence of an endogenous polypeptide or a portion thereof except that the endogenous polypeptide or portion thereof does not contain an aspartate or glutamate residue at its C-terminus; and
(ii) the second polypeptide comprises an amino acid sequence that corresponds to the amino acid sequence of an endogenous polypeptide or a portion thereof which contains a functional group at an equivalent position to the functional group in the second polypeptide.

20. The polypeptide of claim 17 or product of claim 18 or 19, wherein the covalent bond is an amide bond.

21. The polypeptide of claim 17 or 20 or product of any one of claims 18 to 20, wherein the functional group is an amine.

22. A pharmaceutical composition comprising:
(a)(1) a chimeric protein comprising:
(i) a domain comprising the first polypeptide; and
(ii) a domain comprising a self-processing module that contains an N-terminal dipeptide of aspartate or glutamate and proline (D/E-P),
wherein (i) and (ii) are linked by a peptide bond between the aspartate or glutamate residue at the N-terminus of (ii) and the amino acid at the C-terminus of (i) and wherein the self-processing module cleaves the peptide bond between the proline residue and the aspartate or glutamate residue under suitable conditions;
(2) a polypeptide comprising an anhydride group on a C-terminal aspartate or glutamate residue, wherein the aspartate or glutamate residue in the polypeptide is not present at the equivalent position in the amino acid sequence of the corresponding endogenous polypeptide or portion thereof (e.g. obtained by the method of any one of claim 3, 4, 9 or 10);
(3) a composition as defined in claim 16;
(4) a polypeptide as defined in claim 17, 20, or 21; or
(5) a product as defined in any one of claims 18 to 21; and
(b) one or more pharmaceutically acceptable excipients and/or diluents.

23. A pharmaceutical composition as defined in claim 22 for use in therapy or diagnosis.

24. A method of treating a disease in a subject comprising administering to a subject in need thereof a therapeutically effective amount of a pharmaceutical composition of claim 22, thereby treating the disease.

25. The use, method or pharmaceutical composition of any preceding claim, wherein the chimeric protein comprises N-terminus to C-terminus:
(i) a domain comprising a polypeptide; and
(ii) a domain comprising a self-processing module comprising:
(1) an amino acid sequence as set forth in any one of SEQ ID NOs: 1-4;
(2) a portion of (1) comprising an amino acid sequence as set forth in any one of SEQ ID NOs: 5-8;
(3) an amino acid sequence with at least 60% sequence identity to an amino acid sequence as set forth in any one of SEQ ID NOs: 1-4; or
(4) a portion of (3) comprising an amino acid sequence with at least 60% sequence identity to an amino acid sequence as set forth in any one of SEQ ID NOs: 5-8,
wherein the first (N-terminal) amino acid of the domain comprising a self-processing module is an aspartate or glutamate and the second amino acid of the domain comprising a self-processing module is proline;
and wherein the self-processing module cleaves the peptide bond between the first and second amino acids of the domain comprising a self-processing module under suitable conditions.

26. The use, method or pharmaceutical composition of claim 25, wherein the chimeric protein further comprises a linker between (i) and (ii), preferably wherein the linker comprises the motif X₁X₂X₃, wherein:
(a) X₁ and X₂ are independently selected from any amino acid, preferably G and S; and
(b) X₃ is selected from R, N, Q, F, V, H, Y or W, preferably V, H, Y or W.

27. A chimeric protein comprising N-terminus to C-terminus:
(i) a domain comprising a polypeptide;
(ii) a domain comprising a linker; and
(iii) a domain comprising a self-processing module comprising:
(1) an amino acid sequence as set forth in any one of SEQ ID NOs: 1-4;
(2) a portion of (1) comprising an amino acid sequence as set forth in any one of SEQ ID NOs: 5-8;
(3) an amino acid sequence with at least 60% sequence identity to an amino acid sequence as set forth in any one of SEQ ID NOs: 1-4; or
(4) a portion of (3) comprising an amino acid sequence with at least 60% sequence identity to an amino acid sequence as set forth in any one of SEQ ID NOs: 5-8,
wherein the first (N-terminal) amino acid of the domain comprising a self-processing module is an aspartate or glutamate and the second amino acid of the domain comprising a self-processing module is proline;
and wherein the self-processing module cleaves the peptide bond between the first and second amino acids of the domain comprising a self-processing module under suitable conditions.

28. The chimeric protein of claim 27, wherein the linker comprises the motif X₁X₂X₃, wherein:
(a) X₁ and X₂ are independently selected from any amino acid, preferably G and S; and
(b) X₃ is selected from R, N, Q, F, V, H, Y or W, preferably V, H, Y or W.

29. The chimeric protein of claim 27 or 28, wherein the self-processing module comprises:
(1) an amino acid sequence as set forth in SEQ ID NO: 1;
(2) a portion of (1) comprising an amino acid sequence as set forth in SEQ ID NO: 5;
(3) an amino acid sequence with at least 80% sequence identity to an amino acid sequence as set forth in SEQ ID NO: 1 or 2; or
(4) a portion of (3) comprising an amino acid sequence with at least 80% sequence identity to an amino acid sequence as set forth in SEQ ID NO: 5 or 6,
wherein the amino acid sequence comprises aspartate or glutamate at position 1, proline at position 2 and one or more of the following:
(1) alanine at position 17;
(2) alanine at position 23;
(3) arginine at position 28;
(4)glutamine at position 30;
and wherein the self-processing module cleaves the peptide bond between the first and second amino acids of the domain comprising a self-processing module under suitable conditions.

30. The chimeric protein of any one of claims 27 to 29, wherein the self-processing module comprises:
(1) an amino acid sequence as set forth in SEQ ID NO: 1;
(2) a portion of (1) comprising an amino acid sequence as set forth in SEQ ID NO: 5;
(3) an amino acid sequence with at least 99% sequence identity to an amino acid sequence as set forth in SEQ ID NO: 1; or
(4) a portion of (3) comprising an amino acid sequence with at least 99% sequence identity to an amino acid sequence as set forth in SEQ ID NO: 5,
wherein the amino acid sequence comprises aspartate or glutamate at position 1 and proline at position 2;
and wherein the self-processing module cleaves the peptide bond between the first and second amino acids of the domain comprising a self-processing module under suitable conditions.

31. Use of a chimeric protein as defined in any one of claims 27 to 30 to isolate (e.g. purify) a desired polypeptide, wherein the polypeptide in domain (i) of the chimeric protein is the desired polypeptide.

32. A method of isolating (e.g. purifying) a desired polypeptide comprising:
a) providing a sample comprising a chimeric protein of any one of claims 27 to 30, wherein the polypeptide in domain (i) of the chimeric protein is the desired polypeptide;
b) contacting the sample of a) with a solid support under conditions that enable said chimeric protein to selectively bind to said solid support, thereby forming a non-covalent complex between said chimeric protein and the solid support;
c) washing the solid support with a buffer;
d) inducing the self-processing module to cleave the peptide bond between the proline residue and the aspartate or glutamate residue (i.e. between residues 1 and 2) to release the desired polypeptide; and
e) separating the desired polypeptide from the solid support.

33. The chimeric protein of any one of claims 27 to 30, wherein the polypeptide in domain (i) of the chimeric protein is a growth factor, cytokine, chemokine or a portion or derivative thereof.

34. The chimeric protein of claim 33, wherein the growth factor, cytokine or chemokine is selected from any one of TGFα, epigen, epiregulin, EGF, HB-EGF, TGFβ, TNFα, IL1RA, IL-1β, IL-2, IL-4, IL-5, IL-6, IL-7, IL-8 (CXCL8), IL-9, IL-10, IL-12, IL-13, IL-15, IL-17, CCL11, BasicFGF, G-CSF, GM-CSF, INFα, INFγ, CXCL10, CCL2, CCL3, CCL4, PDGF-β, CCL5, VEGF or a functional portion or derivative thereof, preferably TGFα or a functional portion or derivative thereof.

35. The chimeric protein of claim 33 or 34, wherein the polypeptide in domain (i) of the chimeric protein comprises an amino acid sequence as set forth in SEQ ID NO: 17.

36. The chimeric protein of any one of claims 33 to 35, wherein the chimeric protein comprises an amino acid sequence as set forth in SEQ ID NO: 16.

37. A nucleic acid molecule encoding the chimeric protein of any one of claims 27 to 30 or 33 to 36.


SEQUENCE LISTING (17 sequences)

SEQ ID NO: 1 - protein, 244 aa, Neisseria meningitidis
DPLALDLDGDGIETVAAKGFAGALFDHRNQGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSRFKDKVELTAEQAKAANLAGIGRLRDLREAAALSGDLANMLKAYSAAETKEAQLALLDNLIHKWAETD

SEQ ID NO: 2 - protein, 244 aa, Neisseria meningitidis
DPLALDLDGDGIETVATKGFAGSLFDHTNNGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSRFKDKVELTAEQAKAANLAGIGRLRDLREAAALSGDLANMLKAYSAAETKEAQLALLDNLIHKWAETD

SEQ ID NO: 3 - protein, 244 aa, Alysiella filiformis
DPLALDLDGNGIQTTATAGFSGSLFDHNKDGIRTATGWVASGDGLLVRDLNGNGIIDNGGELFGENTLLADGTLAQHGYAALAELDSNADGVVDANDAAFATLRVWQDKNQDGISQADELHTLADLGIQSLNTAYEDVNQDLGNGNSIAQLGSYTKTDGSTAEMADLLFHNDHLYSRFAERIELTAEQSRAANLSGIGRVRDLREAAALFGDLSATLQSYSQADTKQVQMALLDKLVQKWAETD

SEQ ID NO: 4 - protein, 243 aa, Kingella negevensis
DPLILDLDGKGIQTLAPSSISARFDHNADGIATATGWAAAGNGILALDLDNNGKIDSGKEIFGNHSVLSNGTAAAHGYAALAELDSNADGIISALDDTFSSLKVWQDINQDGISQSNELFTLQALGIQSLNLEHQENSKDLGNGNRLTHIGSYTKTDGTTGEMGDVEFASNSLYSRYTDTIELTPEQLQAPNLHGTGRLRDLREAAALSTGLAEILKQYSAAQTKEEQTALLSELVAKWGATD

SEQ ID NO: 5 - protein, 177 aa, Neisseria meningitidis
DPLALDLDGDGIETVAAKGFAGALFDHRNQGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSR

SEQ ID NO: 6 - protein, 177 aa, Neisseria meningitidis
DPLALDLDGDGIETVATKGFAGSLFDHTNNGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSR

SEQ ID NO: 7 - protein, 177 aa, Alysiella filiformis
DPLALDLDGNGIQTTATAGFSGSLFDHNKDGIRTATGWVASGDGLLVRDLNGNGIIDNGGELFGENTLLADGTLAQHGYAALAELDSNADGVVDANDAAFATLRVWQDKNQDGISQADELHTLADLGIQSLNTAYEDVNQDLGNGNSIAQLGSYTKTDGSTAEMADLLFHNDHLYSR

SEQ ID NO: 8 - protein, 176 aa, Kingella negevensis
DPLILDLDGKGIQTLAPSSISARFDHNADGIATATGWAAAGNGILALDLDNNGKIDSGKEIFGNHSVLSNGTAAAHGYAALAELDSNADGIISALDDTFSSLKVWQDINQDGISQSNELFTLQALGIQSLNLEHQENSKDLGNGNRLTHIGSYTKTDGTTGEMGDVEFASNSLYSR

SEQ ID NO: 9 - protein, 399 aa, Artificial sequence
  AP-GSS-His6-OAZ (95-219)-GVY-SEQ ID NO 1
MGSGLNDIFEAQKIEWHEGSSHHHHHHFYSDDRLNVTEELTSNDKTRILNVQSRLTDAKRINWRTVLSGGSLYIEIPGGALPEGSKDSFAVLLEFAEEQLRADHVFIAFHKNREDRAALLRTFSFLGFEIVRPGHPLVPKRPDACFMAYTFEGVYDPLALDLDGDGIETVAAKGFAGALFDHRNQGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSRFKDKVELTAEQAKAANLAGIGRLRDLREAAALSGDLANMLKAYSAAETKEAQLALLDNLIHKWAETD

SEQ ID NO: 10 - protein, 399 aa, Artificial sequence
  AP-GSS-His6-OAZ (95-219)-GVY-SEQ ID NO 2
MGSGLNDIFEAQKIEWHEGSSHHHHHHFYSDDRLNVTEELTSNDKTRILNVQSRLTDAKRINWRTVLSGGSLYIEIPGGALPEGSKDSFAVLLEFAEEQLRADHVFIAFHKNREDRAALLRTFSFLGFEIVRPGHPLVPKRPDACFMAYTFEGVYDPLALDLDGDGIETVATKGFAGSLFDHTNNGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSRFKDKVELTAEQAKAANLAGIGRLRDLREAAALSGDLANMLKAYSAAETKEAQLALLDNLIHKWAETD

SEQ ID NO: 11 - protein, 399 aa, Artificial sequence
  AP-GSS-His6-OAZ (95-219)-GIV-SEQ ID NO 3
MGSGLNDIFEAQKIEWHEGSSHHHHHHFYSDDRLNVTEELTSNDKTRILNVQSRLTDAKRINWRTVLSGGSLYIEIPGGALPEGSKDSFAVLLEFAEEQLRADHVFIAFHKNREDRAALLRTFSFLGFEIVRPGHPLVPKRPDACFMAYTFEGIVDPLALDLDGNGIQTTATAGFSGSLFDHNKDGIRTATGWVASGDGLLVRDLNGNGIIDNGGELFGENTLLADGTLAQHGYAALAELDSNADGVVDANDAAFATLRVWQDKNQDGISQADELHTLADLGIQSLNTAYEDVNQDLGNGNSIAQLGSYTKTDGSTAEMADLLFHNDHLYSRFAERIELTAEQSRAANLSGIGRVRDLREAAALFGDLSATLQSYSQADTKQVQMALLDKLVQKWAETD

SEQ ID NO: 12 - protein, 398 aa, Artificial sequence
  AP-GSS-His6-OAZ (95-219)-GGY-SEQ ID NO 4
MGSGLNDIFEAQKIEWHEGSSHHHHHHFYSDDRLNVTEELTSNDKTRILNVQSRLTDAKRINWRTVLSGGSLYIEIPGGALPEGSKDSFAVLLEFAEEQLRADHVFIAFHKNREDRAALLRTFSFLGFEIVRPGHPLVPKRPDACFMAYTFEGGYDPLILDLDGKGIQTLAPSSISARFDHNADGIATATGWAAAGNGILALDLDNNGKIDSGKEIFGNHSVLSNGTAAAHGYAALAELDSNADGIISALDDTFSSLKVWQDINQDGISQSNELFTLQALGIQSLNLEHQENSKDLGNGNRLTHIGSYTKTDGTTGEMGDVEFASNSLYSRYTDTIELTPEQLQAPNLHGTGRLRDLREAAALSTGLAEILKQYSAAQTKEEQTALLSELVAKWGATD

SEQ ID NO: 13 - protein, 153 aa, Artificial sequence
  AP-GSS-His6-OAZ (95-219)
MGSGLNDIFEAQKIEWHEGSSHHHHHHFYSDDRLNVTEELTSNDKTRILNVQSRLTDAKRINWRTVLSGGSLYIEIPGGALPEGSKDSFAVLLEFAEEQLRADHVFIAFHKNREDRAALLRTFSFLGFEIVRPGHPLVPKRPDACFMAYTFEG

SEQ ID NO: 14 - protein, 246 aa, Neisseria meningitidis
VYDPLALDLDGDGIETVATKGFAGSLFDHTNNGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSRFKDKVELTAEQAKAANLAGIGRLRDLREAAALSGDLANMLKAYSAAETKEAQLALLDNLIHKWAETD

SEQ ID NO: 15 - protein, 6 aa, Artificial sequence
  Linker
GSGESG

SEQ ID NO: 16 - protein, 314 aa, Artificial sequence
  TGFalpha-GSY-SPM-His6-Ctag
MVVSHFNDCPDSHTQFCFHGTCRFLVQEDKPACVCHSGYVGARCEHADLLAGSYDPLALDLDGDGIETVATKGFAGSLFDHTNNGIRTATGWVSADDGLLVRDLNGNGIIDNGAELFGDNTKLADGSFAKHGYAALAELDSNGDNIINAADAAFQTLRVWQDLNQDGISQANELRTLEELGIQSLDLAYKDVNKNLGNGNTLAQQGSYTKTDGTTAKMGDLLLAADNLHSRFKDKVELTAEQAKAANLAGIGRLRDLREAAALSGDLANMLKAYSAAETKEAQLALLDNLIHKWAETDGSSHHHHHHSSGEPEA

SEQ ID NO: 17 - protein, 51 aa, Homo sapiens
MVVSHFNDCPDSHTQFCFHGTCRFLVQEDKPACVCHSGYVGARCEHADLLA